(19) United States
(12) Patent Application Publication
(10) Pub. No.: US 2024/0058034 A1
(43) Pub. Date: Feb. 22, 2024

(54) MULTI-LUMEN CARDIAC ACCESS

(71) Applicant: EDWARDS LIFESCIENCES CORPORATION, Irvine, CA (US)

(72) Inventors: Felino V. Cortez, JR., Bowie, MD (US); Stephen Cournane, Severn, MD (US); Stephen Epstein, Millersville, MD (US); Nancy Ling Chung, Edgewater, MD (US); Luke Anthony Zanetti, Parkton, MD (US); Ashley Nicolette Hinga (formerly Keffer), Eldersburg, MD (US); Wesley Paul Nicholson, Sunderland, MD (US); Sai Prasad Uppalapati, Plano, TX (US)

(21) Appl. No.: 18/452,438

(22) Filed: Aug. 18, 2023

Related U.S. Application Data
(63) Continuation of application No. PCT/US22/16725, filed on Feb. 17, 2022.
(60) Provisional application No. 63/151,553, filed on Feb. 19, 2021.

(51) Int. Cl. A61B 17/34; A61B 17/00

(52) U.S. Cl. CPC A61B 17/3415; A61B 17/00234; A61B 17/3423; A61B 2017/00243; A61B 2017/3425

(57) ABSTRACT

An introducer device includes a proximal hub including a first port and a second port, first and second fluid valves associated with the first port and the second port, respectively, a first elongate lumen projecting distally from the proximal hub, the first elongate lumen being in fluid communication with the first port, and a second elongate lumen projecting distally from the proximal hub, the second elongate lumen being in fluid communication with the second port and fluidly isolated from the first elongate lumen.

### CROSS-REFERENCE TO RELATED APPLICATIONS

This application is a continuation of International Patent Application No. PCT/US2022/016725, filed Feb. 17, 2022, which claims the benefit of U.S. Patent Application No. 63/151,553, filed Feb. 19, 2021, the entire disclosures all of which are incorporated by reference for all purposes.

### BACKGROUND

### Technical Field

The present disclosure generally relates to the field of medical devices and processes.

### Description of Related Art

With respect to certain medical procedures, access to the target treatment site in the patient anatomy can be achieved using various access paths. The timing and routing of such access can have an effect on patient comfort and/or procedure efficacy.

### SUMMARY

Described herein are methods and devices that facilitate execution of certain cardiac and/or other medical procedures by providing for concurrent access to the target patient anatomy for a plurality of medical instruments through a plurality of access lumens, such as lumens of a multi-lumen transapical introducer device.

In some implementations, the present disclosure relates to an introducer device comprising a proximal hub including a first port and a second port, first and second fluid valves associated with the first port and the second port, respectively, a first elongate lumen projecting distally from the proximal hub, the first elongate lumen being in fluid communication with the first port, and a second elongate lumen projecting distally from the proximal hub, the second elongate lumen being in fluid communication with the second port and fluidly isolated from the first elongate lumen.

The first elongate lumen and the second elongate lumen can be part of an elongate access sheath structure.

In some examples, the introducer device further comprises an outer tube surrounding at least a portion of the first elongate lumen and the second elongate lumen.

The proximal hub of the introducer device can further include a third port. For example, in some examples, the first and second ports are configured to receive catheters and the third port is configured to receive a dilator.

In some examples, the proximal hub further includes a proximal flange and a valve seal housing, the valve seal housing houses the first and second fluid valves, and the first and second elongate lumens project distally from the valve seal housing.

The proximal hub may further include a fluid inlet port. The first and second elongate lumens may be fluidly isolated from one another. In some examples, distal openings of the first and second elongate lumens are tapered towards one another.

The introducer device can comprise a first elongate shaft that forms the first elongate lumen, a second elongate shaft that forms the second elongate lumen, and an outer tube disposed around a length of the first and second elongate lumens.

In some implementations, the present disclosure relates to an introducer device comprising a proximal hub including a first valved port and a second valved port, and an elongate access sheath structure that projects distally from the proximal hub, the elongate access sheath structure comprising a first elongate lumen in fluid communication with the first port and a second elongate lumen in fluid communication with the second port and fluidly isolated from the first elongate lumen.

The elongate access sheath structure can comprise a first elongate shaft forming the first elongate lumen and a second elongate shaft forming the second elongate lumen. For example, the elongate access sheath structure can further comprise an outer tube, at least a portion of the first elongate shaft and the second elongate shaft being disposed within the outer tube. The first and second elongate shafts may extend past a distal end of the outer tube. In some examples, distal ends of the first and second elongate shafts are tapered towards a central axis of the outer tube.

The proximal hub can further comprise a third valved port. For example, the third valved port may be positioned about a central axis of the elongate access sheath structure.

In some examples, the elongate access sheath structure has an oval cross-sectional shape. The introducer device may comprise a tear-away sheath covering a distal end of the elongate access sheath structure. The first and second valved ports may include respective hemostatic fluid valves.

In some implementations, the present disclosure relates to a method of treating a heart valve. The method comprises inserting a multi-lumen introducer device into a chamber of a heart, securing the multi-lumen introducer to the heart, advancing an imaging device into the chamber through a first lumen of the multi-lumen introducer device, advancing a working instrument into the chamber through a second lumen of the multi-lumen introducer device, while generating imaging data within the heart using the imaging device, performing a cardiac procedure within the heart using the working instrument, and withdrawing the working instrument through the second lumen of the introducer.

The working instrument may comprise an elongate shaft having an atraumatic end effector associated with a distal end thereof. The method can further comprise contacting a leaflet of a valve of the heart with the end effector and deploying a tissue anchor from the distal end of the elongate shaft through the leaflet.

In some examples, the method comprises puncturing an apex of the heart with a needle, inserting a guidewire into the chamber through the needle, removing the needle from off of the guidewire, advancing a dilator over the guidewire to dilate an opening in the apex of the heart, removing the dilator, and advancing the multi-lumen introducer device into the chamber over the guidewire.

In some examples, the working instrument comprises an ablation catheter. For example, the method may comprise advancing the ablation catheter through a mitral valve of the heart and into a left atrium of the heart and ablating cardiac tissue around a pulmonary vein ostium of the heart using the ablation catheter.

The method may further comprise, prior to said inserting the multi-lumen introducer device and advancing the working instrument through the second lumen of the multi-lumen introducer device, advancing a dilator through the multi-lumen introducer device. For example, the dilator may be advanced through the second lumen of the multi-lumen introducer device. Alternatively, the dilator may be advanced through a third lumen of the multi-lumen introducer device. the third lumen may be disposed between the first lumen and the second lumen.

Each method disclosed herein also encompass simulations of the method, which are useful, for example, for teaching, demonstration, testing, device development, and procedure development. For example, methods for treating or diagnosing a patient include corresponding simulated methods performed on simulated patients. Suitable simulated patients or anthropogenic ghosts can include any combination of physical and virtual elements. Examples of physical elements include whole human or animal cadavers, or any portion thereof, including, organ systems, individual organs, or tissue; and manufactured cadaver, organ system, organ, or tissue simulations. Examples of virtual elements include visual simulations, which can be displayed on a screen; projected on a screen, surface, or volume; and holographic images. The simulation can also include one or more of another type of sensory input, for example, auditory, tactile, and olfactory stimuli.

For purposes of summarizing the disclosure, certain aspects, advantages and novel features are described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a cutaway view of a human heart showing certain catheter access paths and detailed images of certain anatomy thereof in accordance with one or more examples.

FIG. 2 is a cutaway view of a heart having a transapical introducer device disposed to provide access to one or more chambers of the heart by a heart valve repair device disposed at least partially within a lumen of the introducer in accordance with one or more examples.

FIG. 3 shows a side view of certain imaging devices disposed in positions to generate echo images of cardiac anatomy of a patient in accordance with one or more examples.

FIGS. 4A-4D show isometric, proximal end, side, and cross-sectional side views, respectively, of a multi-lumen introducer device in accordance with one or more examples.

FIGS. 5A and 5B show proximal end and cross-sectional side views, respectively, of a multi-lumen introducer device in accordance with one or more examples.

FIGS. 6A and 6B shows perspective and cutaway views, respectively, of a heart having a multi-lumen introducer device disposed at least partially therein in accordance with one or more examples.

FIG. 7 is a cutaway view of a heart having a multi-lumen introducer disposed at least partially therein, with a plurality of medical instruments accessing one or more chambers of the heart through respective lumens of the multi-lumen introducer device in accordance with one or more examples.

FIG. 8 is a cutaway view of a heart having a multi-lumen introducer disposed at least partially therein, with a plurality of medical instruments accessing one or more chambers of the heart through respective lumens of the multi-lumen introducer device in accordance with one or more examples.

FIGS. 9-1, 9-2, 9-3, 9-4, and 9-5 provide a flow diagram illustrating a process for performing a medical procedure in accordance with one or more examples.

FIGS. 10-1, 10-2, 10-3, 10-4, and 10-5 provide cross-sectional images of cardiac anatomy and certain devices/systems corresponding to operations of the process of FIGS. 9-1, 9-2, 9-3, 9-4, and 9-5 according to one or more examples.

FIGS. 11A and 11B show a multi-lumen introducer device implemented with a tearaway sheath in accordance with one or more examples.

To further clarify various aspects of examples of the present disclosure, a more particular description of certain examples will be made by reference to various aspects of the appended drawings. It is appreciated that these drawings depict only typical examples of the present disclosure and are therefore not to be considered limiting of the scope of the disclosure. Moreover, while the figures can be drawn to scale for some examples, the figures are not necessarily drawn to scale for all examples. Examples of the present disclosure will be described and explained with additional specificity and detail through the use of the accompanying drawings.

### DETAILED DESCRIPTION

The following description refers to the accompanying drawings, which illustrate specific examples. Other examples having different structures and operation do not depart from the scope of the disclosure.

The following includes a general description of human cardiac anatomy that is relevant to certain inventive features and examples disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

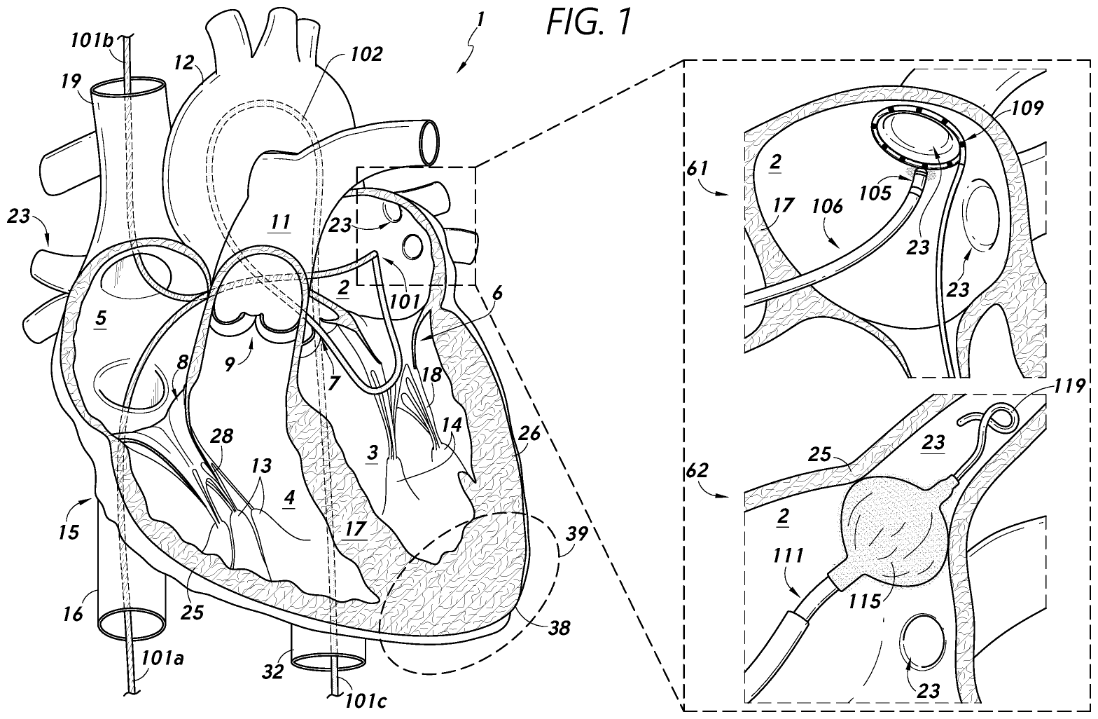

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle 17, referred to as the septum, separates the left 2 and right 5 atria and the left 3 and right 4 ventricles. The inferior tip 38 of the heart 1 is referred to as the apex and is generally located on the midclavicular line, in the fifth intercostal space. The apex 19 can be considered part of the greater apical region 39 (referred to in some contexts herein as simply the “apex”). References herein to the apex of a heart may be understood to reference the apex or the apical region generally.

The left ventricle 3 is the primary pumping chamber of the heart 1. A healthy left ventricle is generally conical or apical in shape in that it is longer (along a longitudinal axis extending in a direction from the aortic valve 7 to the apex 38) than it is wide (along a transverse axis extending between opposing walls 25, 26 at the widest point of the left ventricle) and descends from a base 15 with a decreasing cross-sectional circumference to the point or apex 38. Generally, the apical region 39 of the heart is a bottom region of the heart that is within the left or right ventricular region but is distal to the mitral 6 and tricuspid 8 valves and toward the tip 38 of the heart. More specifically, the apical region 39 may be considered to be within about 20 cm to the right or to the left of the median axis 27 of the heart 1.

The pumping of blood from the left ventricle 3 is accomplished by a squeezing motion and a twisting or torsional motion. The squeezing motion occurs between the lateral wall 26 of the left ventricle 3 and the septum 17. The twisting motion is a result of heart muscle fibers that extend in a circular or spiral direction around the heart. When these fibers contract, they produce a gradient of angular displacements of the myocardium from the apex 38 to the base 15 about the longitudinal axis of the heart. The resultant force vectors extend at angles from about 30-60 degrees to the flow of blood through the aortic valve 7. The contraction of the heart is manifested as a counterclockwise rotation of the apex 38 relative to the base 15, when viewed from the apex 38. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae (13, 16) and papillary muscles (10, 15) for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles 14. The papillary muscles 14 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles 13, 14 and the chordae tendineae 28, 18 attached thereto, which can elongate and/or rupture. If a valve is functioning properly, when closed, the free margins or edges of the leaflets come together and form a tight junction, the are of which, in the mitral valve, is known as the line, plane or area of coaptation. Normal mitral and tricuspid valves open when the respective associated ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

### Cardiac Intervention: Tissue Ablation

In certain patients or individuals, various conditions and/or events can result in compromised cardiac electrical flow, causing the development and/or occurrence of an abnormal heart rhythm. For example, atrial fibrillation is a relatively common cardiac condition associated with abnormal electrical flow and/or heart rhythm characterized by relatively rapid and irregular beating of the atria. In some cases, mitral valve disease can be associated with atrial fibrillation. Generally, atrial fibrillation may be present in up to 50% of patients undergoing mitral valve surgery. The presence of atrial fibrillation in a patient undergoing a cardiac intervention can contribute to an increased risk of systemic embolization, anticoagulant-related hemorrhage, and mortality.

When atrial fibrillation occurs, the normal regular electrical impulses generated by the sinoatrial (SA) node 21 in the right atrium 5 may become overwhelmed by disorganized electrical impulses, which may lead to irregular conduction of ventricular impulses that generate the heartbeat. Atrial fibrillation generally originates in the right atrium 5, that where conduction path disturbances begin, and can present in the left atrium 2 as aberrant electrical signals propagate in the left atrial tissue, such as in the area of one or more of the pulmonary veins 23.

Various pathologic developments can lead to, or be associated with, atrial fibrillation. For example, progressive fibrosis of the atria may contribute at least in part to atrial fibrillation. The formation of fibrous tissue associated with fibrosis can disrupt or otherwise affect the electrical pathways of the cardiac electrical system due to interstitial expansion associated with tissue fibrosis. In addition to fibrosis in the muscle mass of the atria, fibrosis may also occur in the sinoatrial node and/or atrioventricular node, which may lead to atrial fibrillation.

Fibrosis of the atria may be due to atrial dilation, or stretch, in some cases. Dilation of the atria can be due to a rise in the pressure within the heart, which may be caused by excessive atrial fluid pressures in some cases, and/or may be due to a structural abnormality in the heart, such as valvular heart disease (e.g., mitral stenosis, mitral regurgitation, tricuspid regurgitation), hypertension, congestive heart failure, or other condition. Dilation of the atria can lead to the activation of the renin aldosterone angiotensin system (RAAS), and subsequent increase in matrix metalloproteinases and disintegrin, which can lead to atrial remodeling and fibrosis and/or loss of atrial muscle mass.

In addition to atrial dilation, inflammation in the heart can cause fibrosis of the atria. For example, inflammation may be due to injury associated with a cardiac surgery, such as a valve repair operation, or the like. Alternatively, inflammation may be caused by sarcoidosis, autoimmune disorders, or other condition. Other cardiovascular factors that may be associated with the development of atrial fibrillation include high blood pressure, coronary artery disease, mitral stenosis (e.g., due to rheumatic heart disease or mitral valve prolapse), mitral regurgitation, hypertrophic cardiomyopathy (HCM), pericarditis, and congenital heart disease. Additionally, lung diseases (such as pneumonia, lung cancer, pulmonary embolism, and sarcoidosis) may contribute to the development of atrial fibrillation in some patients

Atrial fibrillation is associated with increased morbidity and mortality. Generally, it can be advisable for patients with pre-existing atrial fibrillation who undergo mitral valve surgery to have surgical ablation performed in connection with such valve surgery to treat the atrial fibrillation. Treatment of mitral valve insufficiency and/or certain other cardiac pathologies without addressing atrial fibrillation can result in poor outcomes. For example, atrial fibrillation is considered, along with age and left ventricular dysfunction, one of the factors influencing late survival after mitral repair interventions.

Certain surgical and minimally-invasive tissue ablation techniques can be implemented as a means to restore normal sinus rhythm to a heart experiencing atrial fibrillation. For example, as shown in detailed image 61, cardiac tissue ablation may be implemented by delivering energy, such as radiofrequency electromagnetic radiation energy, through a catheter 106 or other transducer device to target areas in the heart at which an arrhythmia originates. Such energy can ablate or destroy relatively small focal areas of the cardiac tissue and disrupt the abnormal electrical activity occurring therein. According to some solutions, ablation can be implemented using minimally invasive techniques, such as through transcatheter access to the target atrium and/or other area of the heart.

In addition to radiofrequency radiation ablation, other types of catheter ablation can be implemented. For example, as shown in detailed image 62, cardiac tissue ablation may be implemented using cryoablation, which generally utilizes a pressurized refrigerant in a catheter tip or other device to ablate the relevant source of arrhythmia. Some such solutions involve the use of a cryoballoon device 115, wherein the cryoballoon can be used to cool/freeze the targeted tissue using coolant inside the balloon 115 to alter abnormal electrical activity.

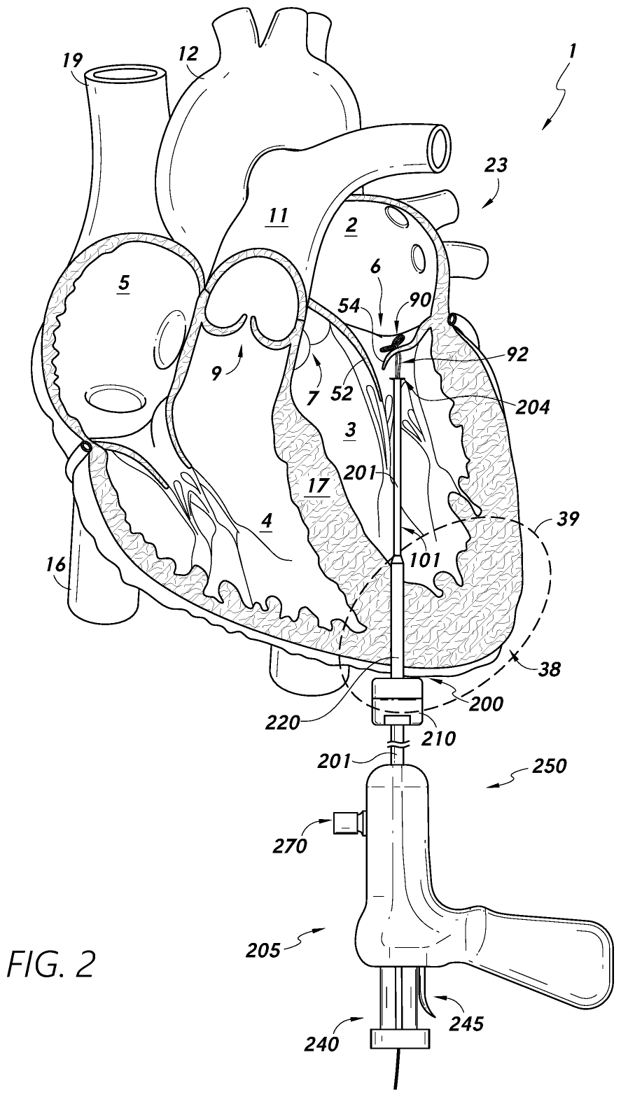

For patients that suffer from atrial fibrillation, it can generally be advisable to perform tissue ablation at the time of mitral surgery. For example, When a patient undergoes a valve replacement procedure, or another procedure designed to correct for degenerative mitral regurgitation, such as through leaflet tethering as may be implemented using a valve repair system as described below in connection with FIG. 2 and various other figures and examples associated with the present disclosure, cardiac tissue ablation may desirably be performed in connection with such procedures in order to treat atrial fibrillation and improve patient outcomes. For example, as many as 35% of patients undergoing mitral valve procedures may suffer from atrial fibrillation. In such cases, it may be desirable to ablate cardiac tissue within the left atrium, right atrium, around one or more pulmonary veins, and/or other areas of the heart. The targeted ablation tissue may be based at least in part on a determination of how long or short the relevant atrial fibrillation is.

According to some solutions, cardiac tissue ablation may be performed in connection with an open-heart surgical operation. Alternatively, access to the left atrium 2 or other area of the heart for the purpose of performing tissue ablation, as described above, can be achieved using a transcatheter approach, wherein an ablation catheter may be delivered to the target chamber (e.g., left atrium) through the vasculature of the patient, such as through one or more arteries or veins of the arm, groin, or neck. For example, as shown by the catheter 101a, access to the left atrium 2 may be made via the inferior vena cava 16, which may be accessed via the femoral artery in some implementations. However, access to the left atrium through the femoral artery can require as much as 6 feet or more of catheter length.

FIG. 1 shows various catheters 101 that may be used to implement cardiac tissue ablation, such as within the left atrium 2 or right atrium 5, in accordance with aspects of the present disclosure. The catheters 101 can advantageously be steerable and relatively small in cross-sectional profile to allow for traversal of the various blood vessels and chambers through which the ablation device may be advanced to, for example, the left atrium or other anatomy or chamber. Catheter access to the left atrium 2 in accordance with certain transcatheter ablation solutions may be made by first accessing the right atrium 5 via the inferior vena cava 16 (as shown by the catheter 101a) or the superior vena cava 19 (as shown by the catheter 101b) and crossing the atrial septum 17 (e.g., in the area at or near the fossa ovalis) to access the left atrium 2.

Although access to the left atrium is illustrated and described in connection with certain examples as being via the right atrium and/or vena cavae, such as through a transfemoral or other transcatheter procedure, other access paths/methods may be implemented in accordance with examples of the present disclosure. For example, transaortic access may be implemented, wherein a delivery catheter 101c is passed through the descending aorta, aortic arch 12, ascending aorta, and aortic valve 7, and into the left atrium 2 through the mitral valve 6.

Access to the left atrium 2 may be desirable for the purpose of allowing for the distal end of the catheter to be brought into proximity with the ostium of one or more of the pulmonary veins 23, which may represent sources of cardiac arrhythmia in some patients. For example, image 61 shows an example detail of the left atrium 2 in which an ablation catheter 106 having an ablation transducer tip 105 is brought into contact and/or close proximity with tissue surrounding the pulmonary vein 23, which may be any of the pulmonary veins associated with the left atrium 2. Radiation may be emitted from the tip 105 to cause damage to the tissue around the pulmonary vein 23. In some implementations, an ablation guide 109 is placed around the pulmonary vein ostium 23 to serve as a guide for passing the distal tip 105 of the ablation catheter 106 around the ostium of the pulmonary vein 23 in a desired path. In some implementations, multiple pulmonary veins are ablated in connection with a single treatment. Ablation of the tissue effectively kills one or more layers of the myocardial tissue. In some contexts, ablation around the pulmonary vein ostium as shown in image 61 may serve to electrically isolate the pulmonary vein 23 to reduce proliferation of aberrant electrical signals through the myocardial tissue.

In cases in which septal crossing through the interatrial septal wall is not possible, other access routes may be taken to the left atrium 2. For example, in patients suffering from a weakened and/or damaged interatrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). In such cases, transaortic or transapical access, such as transapical access using one or more introducer devices as described below in connection with FIG. 2, may be implemented to access the target anatomy.

### Cardiac Intervention: Transapical Valve Repair

FIG. 2 is a cutaway view of a heart 1 having a heart valve repair device 250 disposed at least partially within a chamber thereof in accordance with one or more examples. In some implementations, access can be made to a chamber of the heart, such as a target ventricle (e.g., left ventricle 2) associated with a diseased heart valve (e.g., mitral valve 6), through the apical region 39 of the heart. For example, access into the left ventricle 3 (e.g., to perform a mitral valve repair) may be gained by making a relatively small incision at the apical region 39, close to (or slightly skewed toward the left of) the median axis of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex 38, such as via lateral ventricular wall, a region between the apex 38 and the base of a papillary muscle, or even directly at the base of a papillary muscle. In some implementations, the incision made to access the appropriate ventricle of the heart is no longer than about 1 mm to about 5 cm, from 2.5 mm to about 2.5 cm, or from about 5 mm to about 1 cm in length.

Certain inventive features disclosed herein relate to certain heart valve repair systems and devices, and/or systems, process, and devices for repairing any other type of target organ tissue. In some implementations, a heart valve repair device configured to facilitate placement of certain tissue anchors in biological tissue (e.g., heart valve leaflet tissue) may be employed in repairing a mitral valve in patients suffering from degenerative mitral regurgitation or other condition. In some implementations, a transapical off-pump echo-guided repair procedure is implemented in which at least part (e.g., a shaft portion/assembly 201) of a valve repair system is inserted in the left ventricle 3 and steered to the surface of a diseased portion of a target mitral valve leaflet 54 and used to deploy/implant a tissue anchor 90 in the target leaflet 54.

The tissue anchor 90 (e.g., sutureform formed into a bulky knot) may advantageously be integrated or coupled with one or more artificial/synthetic cords 92 serving a function similar to that of chordae tendinea. Such artificial cord(s) 92 may comprise suture(s) and/or suture tail portions associated with a knot-type tissue anchor and may comprise any suitable or desirable material, such as expanded polytetrafluoroethylene (ePTFE) or the like. The term “suture” is used herein according to its broad and ordinary meaning and may refer to any elongate cord, strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord,” “chordae,” and “suture” may be used substantially interchangeably. In addition, use of the singular form of any of the suture-related terms listed above, including the terms “suture” and “cord,” may be used to refer to a single suture/cord, or to a portion thereof. For example, where a suture knot or anchor is deployed on a distal side of a tissue portion, and where two suture portions extend from the knot/anchor on a proximal side of the tissue, either of the suture portions may be referred to as a “suture” or a “cord,” regardless of whether both portions are part of a unitary suture or cord.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair/treat regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

In some implementations, an introducer device 200 may be utilized to facilitate access to a ventricle of a heart via the apical region of the heart. Compared to transfemoral access to the heart, transapical access via an introducer 200, as shown in FIG. 2, can provide a significantly shorter distance between the access point and the target chamber of the heart (e.g., left atrium 2). For example, as compared with an about 2-meter-long (about 6-foot-long) catheter navigated through the patient's vasculature, the relatively shorter instrument 201 (e.g., shaft of valve repair device), when advanced to the target site within the heart via the transapical introducer device 200 can be relatively easy to control and can result in improved precision and accuracy with respect to targeting the target leaflet 54 or other anatomy within the ventricle 3 or atrium 2. Furthermore, transapical access using an introducer device 200 as shown in FIG. 2 can be preferable to surgical solutions requiring the chest of the patient to be opened to provide access to the heart.

According to some implementations of valve-repair procedures, an incision into the apical region 39 of a ventricle of the heart is made. For convenience, the image of FIG. 2 shows access into the left ventricle 3. An introducer device 200 containing a one or more ports having associated fluid-retention valves to prevent blood loss and/or air entry into the ventricle 3 may be inserted into the site of entry to provide port access thereto. A valved introducer device like that shown in FIG. 2 may be used to provide access to various medical instruments configured to be passed through the access lumen 220 of the introducer 200, such as a catheter, shaft, or the like.

With respect to the valve repair procedure associated with the system illustration of FIG. 2, a shaft 201 of the valve repair device 250 may be inserted into the lumen 201 to provide access to the ventricle 3 and/or atrium 2 for at least the distal end 204 of a shaft of the repair device 250. Once inside the chamber 3, the shaft 201 of the repair device 250 may be advanced through the lumen 220 of the introducer 200. In some examples, a sheath may be inserted through the introducer 200 through which one or more other instruments can be advanced. For instance, an endoscope may first be advanced into the chamber 3 to visualize the ventricle 3, the valve 6, and/or the sub-valvular apparatus. By using an appropriate endoscope, a careful analysis of the malfunctioning valve 6 may be performed. Each segment of each leaflet may be carefully assessed to determine its pliability, integrity, and motion. Based on this assessment, the practitioner can determine whether the valve can indeed be repaired or must be replaced. The motion of the leaflets 52, 54 can be classified as slightly dysfunctional, prolapsed, or restricted and based on this classification, the necessary steps of the repair can be determined.

As referenced above, mitral valve regurgitation generally increases the workload on the heart and may lead to very serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 36 is particularly problematic and often life threatening. Methods and devices are provided herein, as well as in the '761 PCT Application and the '170 PCT Application, for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. Various procedures can be performed in accordance with the methods described therein to effectuate a cardiac valve repair, which may depend on the specific abnormality and the tissues involved.

To provide access to the heart through the chest for introducer placement, one or more incisions may be made proximate to the thoracic cavity to provide a surgical field of access. The total number and length of the incisions to be made can depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision of, for example, approximately 5 cm or less made in the skin of the body. The incision may be vertical, horizontal, or slightly curved. If the incision is located along one or more ribs, it may advantageously follow the outline of the rib in some cases. The opening may advantageously extend deep enough to allow access to the thoracic cavity between the ribs or under the sternum and is preferably set close to the rib cage and/or diaphragm, dependent on the entry point chosen.

The valve repair device 250 includes a rigid elongate tube 201 forming at least one internal working lumen. Although described in certain examples and/or contexts as comprising a rigid elongate tube, it should be understood that tubes, shafts, lumens, conduits, and the like disclosed herein may be either rigid, at least partially rigid, flexible, and/or at least partially flexible. Therefore, any such component described herein, whether or not referred to as rigid herein, should be interpreted as possibly being at least partially flexible. In accordance with the present disclosure, the rigid elongate tube 201 may be referred to as a shaft for simplicity. Implementation of a valve-repair procedure utilizing the valve repair device 250 can be performed in conjunction with certain imaging technology designed to provide visibility of the shaft 201 of the valve repair device 250 and surrounding anatomy according to a certain imaging modality, such as echo imaging. Generally, when performing a valve-repair procedure utilizing the valve repair device 250 (also referred to herein as a tissue anchor delivery device/system), the operating physician may advantageously work in concert with an imaging technician, who may coordinate with the physician to facilitate successful execution of the valve-repair procedure.

In addition to the delivery shaft 201, the valve repair device 250 may include a plunger feature 240, which may be used or actuated to manually deploy a pre-formed knot 90, such as a bulky knot as described in detail below. The valve repair device 250 may further include a plunger lock mechanism 245, which may serve as a safety lock that locks the valve delivery system until ready for use or deployment of a leaflet anchor as described herein. The plunger 240 may have associated therewith a suture-release mechanism, which may be configured to lock in relative position a pair of suture tails 92 associated with a pre-formed knot anchor 90 to be deployed. For example, the suture portions 92 may be ePTFE sutures. The device 250 may further comprise a flush port 270, which may be used to de-air the lumen of the shaft 201. For example, heparinized saline flush, or the like, may be connected to the flush port 150 using a female Luer fitting to de-air the valve repair device 250. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure.

The lumen of the shaft 201 may house a needle (not shown) that is initially wrapped at least in part with a pre-formed knot sutureform anchor, as described in detail herein. In some examples, the shaft 201 presents a relatively low profile. For example, the shaft 201 may have a diameter of approximately 3 mm or less (e.g., 9 Fr). The shaft 201 is associated with an atraumatic tip 204 feature. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, or otherwise physically related to the second feature, element, component, device, or member.

The atraumatic tip 204 can serve as a leaflet-positioner component, which may be used for deployment and/or positioning of the suture-type tissue anchor. The atraumatic tip 204, disposed at the distal end of the shaft 201, may be configured to have deployed therefrom a wrapped pre-formed suture knot (e.g., sutureform), as described herein. FIG. 2 shows the knot-type tissue anchor 90 in a deployed configuration. In some examples, the atraumatic tip 204 can have echogenicity-enhancing features/characteristics. The atraumatic tip 204 may be referred to as an “end effector.” In addition to a pre-formed knot sutureform and associated needle, the shaft 201 may be configured to house an elongated knot pusher tube (not shown; also referred to herein as a “pusher”), which may be actuated using the plunger 140 in some examples. As described in further detail below, the tip 204 provides a surface against which the target valve leaflet may be held in connection with deployment of a leaflet anchor. The distal end 204 of the shaft 201 can contact a proximal surface of the mitral valve leaflet 54, without or substantially without damaging the leaflet 54. For example, the end/tip portion or component 204 can have a relatively blunt form or configuration. The end/tip portion or component 204 can be configured to maintain contact with the proximal side of the valve leaflet 54 as the heart beats to facilitate reliable delivery of the anchor 191/190 to the target site on the leaflet 54.

As shown, the repair device 250 may be used to deliver a “bulky knot” type tissue anchor 90. For example, the repair device 250 may be utilized to deliver a tissue anchor (e.g., bulky knot) on a distal side of a mitral valve leaflet. The tip 204 (e.g., “end effector”), can be placed in contact with the ventricular side of a leaflet 54 of a mitral valve. The tip 204 can be coupled to the distal end portion of the shaft 201, wherein the proximal end portion of the shaft 201 may be coupled to a handle portion 205 of the repair device 250, as shown. Generally, the elongate pusher (not shown) may be movably disposed within a lumen of the shaft 201 and coupled to a pusher hub (not shown) that is movably disposed within the handle 120 and releasably coupled to the plunger 240. A needle (not shown) carrying a pre-formed tissue anchor sutureform can be movably disposed within a lumen of the pusher and coupled to a needle hub (not shown) that is also coupled to the plunger 240. The plunger 240 can be used to actuate or move the needle and the pusher during deployment of a distal anchor and can be movably disposed at least partially within the handle 205. For example, the handle 205 may define a lumen in which the plunger 240 can be moved. During operation, the pusher may also move within the lumen of the handle 205. The plunger lock 245 can be used to prevent the plunger 240 from moving within the handle 205 during storage and prior to performing a procedure to deploy a tissue anchor.

The needle may have the pre-formed knot disposed about a distal portion thereof while maintained in the shaft 201. For example, the pre-formed knot may be formed of one or more sutures configured in a coiled sutureform having a plurality of winds/turns around the needle over a portion of the needle that is associated with a longitudinal slot in the needle that runs from the distal end thereof. Although the term “sutureform” is used herein, it should be understood that such components/forms may comprise suture, wire, or any other elongate material wrapped or formed in a desired configuration. The coiled sutureform can be provided or shipped disposed around the needle. In some instances, two suture tails 92 extend from the coiled sutureform 90. The suture tails 92 may extend through the lumen of the needle and/or through a passageway of the plunger 240 and may exit the plunger 240 at a proximal end portion thereof. The coiled sutureform may advantageously be configured to be formed into a suture-type tissue anchor 90 (referred to herein as a “bulky knot”) in connection with an anchor-deployment procedure. The coiled sutureform can be configurable to a knot/deployed configuration as shown in FIG. 2 by approximating opposite ends of the coiled portion thereof towards each other to form one or more loops.

As described herein, the repair device 250 can be used in beating heart mitral valve repair procedures. In some instances, the shaft 201 of the repair device 250 can be configured to extend and contract with the beating of the heart. During systolic contraction, the median axis of the heart generally shortens. For example, the distance from the apex 19 of the heart to the valve leaflets 52, 54 can vary by about 1 centimeter (cm) to about 2 centimeters (cm) with each heartbeat in some patients. In some instances, the length of the shaft 201 that protrudes from the handle 205 can change with the length of the median axis of the heart. That is, distal end of the shaft 201 can be configured to be floating such that the shaft can extend and retract with the beat of the heart so as to maintain contact with the target mitral valve leaflet 54.

Although FIG. 2 shows the repair device 250 being used to deploy a tissue anchor 90 in a posterolateral leaflet 54 of a mitral valve 6, it should be understood that the repair device 250 can also be used to deliver a tissue anchor to the anteromedial mitral valve leaflet, or any other type of valve leaflet or tissue. Although certain examples are presented herein the context of mitral valve repair, it should be understood that the principles disclosed herein are applicable to other valves or biological tissues, such as a tricuspid valve.

The shaft 201 can be configured to facilitate performance of one or more functions, such as grasping, suctioning, irrigating, cutting, suturing, or otherwise engaging a valve leaflet. The distal end, or tip, 204 of the shaft 201 can be configured to contact the mitral valve leaflet 54 without substantially damaging the leaflet to facilitate repair of the valve 6. For example, during a valve-repair procedure, the handle 240 coupled to the shaft 201 can be manipulated in such a manner so that the leaflet 54 is contacted with the functional distal portion of the shaft 201 and a repair effectuated.

Once the tip 204 is positioned in the desired position, the distal end of the shaft 201 and the tip 204 may be used to drape, or “tent,” the leaflet 54 to better secure the tip 204 in the desired position. Draping/tenting may advantageously facilitate contact of the tip 204 with the leaflet 54 throughout one or more cardiac cycles, to thereby provide more secure or proper deployment of leaflet anchor(s). The target location may advantageously be located relatively close to the free edge of the target leaflet 54 to minimize the likelihood of undesirable intra-atrial wall deployment of the anchor. Navigation of the tip 204 to the desired location on the underside of the target valve leaflet 54 may be assisted using echo imaging, as described in detail herein. Echo imaging may be relied upon to confirm correct positioning of the tip 204 prior to anchor/knot deployment.

With the shaft 201 positioned against the target leaflet 54, the plunger 240 of the repair device 250 can be actuated to move a needle and a pusher disposed within the shaft 201, such that the coiled sutureform portion of the suture anchor 90 slides off the needle. As the plunger 240 is actuated, a distal piercing portion of the needle can puncture the leaflet 54 and form an opening in the leaflet. In some instances, the needle (not shown) is projected a distance of between about 5-8 mm, or less, distally beyond the distal end of the shaft 201. In some instances, the needle 130 is projected a distance of between about 3-10 mm. In some instances, the needle 130 is projected a distance of about 2 cm, or greater. In some instances, the needle extends until the distal tip of the needle and the entire coiled sutureform 90, which is wrapped around the needle at such stage, extend through the leaflet 54. While the sutureform wrapped on the needle is projected into the atrial side of the leaflet 54, the shaft 201 and tip 204 advantageously remain entirely on the ventricular side of the leaflet 54.

As the pusher (not shown) within the tissue anchor delivery device shaft 201 is moved distally, a distal end of the pusher advantageously moves or pushes the distal coiled sutureform (e.g., pre-deployment coiled portion of the suture anchor 90) over the distal end of the needle and further within the atrium 2 of the heart on a distal side of the leaflet 54, such that the sutureform extends distally beyond a distal end of the needle. After the sutureform has been pushed off the needle, pulling one or more of the suture tail(s) 92 (e.g., suture strands extending from the coiled portion of the suture) associated with the tissue anchor 90 proximally can cause the sutureform to form a bulky knot anchor 90, as shown in FIG. 2. For example, the bulky knot suture anchor 90 may be formed by approximating opposite ends of the coils of the sutureform towards each other to form one or more loops. After the sutureform has been formed into the bulky knot tissue anchor 90, the repair device 250 can be withdrawn proximally, leaving the tissue anchor 90 disposed on the atrial side of the leaflet 54. In some instances, two suture tails 92 may extend from the proximal/ventricle side of the leaflet 54 and out of the heart 1. For example, the delivery device shaft 201 can be slid/withdrawn over the suture tail(s) 92.

The suture tails 92 coupled to the anchor 90 may be secured at the desired tension using a pledget (not shown) or other suture-fixing/locking device or mechanism on the outside of the heart through which the suture tails 92 may run. Furthermore, a knot or other suture fixation mechanism or device may be implemented to hold the sutures at the desired tension and to such pledget. With the suture tail(s) 92 fixed to the ventricle wall, a ventricular portion of the suture tail(s) 92 may advantageously function as replacement leaflet cords (e.g., chordae tendineae) that are configured to tether the target leaflet 54 in a desired manner.

In certain implementations, testing of location and/or tension of the anchor 90 and/or suture tail(s) 92 may be performed by gently tensioning the suture tails until leaflet motion is felt and/or observed. Echo imaging technology may be used to view and verify the anchor placement and resulting leaflet function. The steps and processes outlined above for placing a suture-knot-type tissue anchor may be repeated as necessary until the desired number of anchors have been implanted on the target valve leaflet. The appropriate number of leaflet anchors may advantageously be determined to produce the desired coaptation of the target valve leaflets 54, 52. In some implementations, one or more leaflet anchors is deployed in each of the mitral valve leaflets, wherein sutures/cords coupled to separate leaflets are secured together in the heart by tying them together with knots or by another suitable attachment device, creating an edge-to-edge repair to decrease the septal-lateral distance of the mitral valve orifice.

Generally, the shaft 201 of the tissue anchor repair device 250 may be slowly advanced into the introducer 200 until the tip 204 has flushed the introducer 200 and entered the ventricle 3. In so doing, it may be desirable to advance the shaft 201 within the ventricle 3 in such a way as to avoid traversing areas populated by papillary muscles and/or associated chordae tendineae to avoid entanglement therewith. In order to facilitate or ensure avoidance of such anatomy, imaging technology may advantageously be implemented to provide at least partial visibility of the shaft 201 within the ventricle 3, as well as certain anatomical features within the ventricle. In some implementations, hybrid imaging technologies may be used, wherein echo imaging is used in combination with a different imaging modality. Multi-imaging modalities may provide improved visibility of anatomical and/or delivery system components.

When relying upon echo imaging, problems and/or difficulties may result from loss of visibility of one or more components of the tissue anchor delivery device, such as loss of visibility of the tip of the anchor-delivery shaft. In such instances, it may be necessary to re-start the anchor-placement procedure. Because the tissue anchor delivery device is inserted through a port in the heart, re-starting delivery of a leaflet anchor can involve additional manipulation on the entry side of the heart, which may result in increased trauma that may have detrimental effects on the cardiac tissue. Examples of the present disclosure advantageously allow for concurrent use of valve repair devices and imaging devices within the heart to improve imaging of the repair device and surrounding anatomy.

The introducer device 200 can include a hub portion 210, wherein the hub 210 is fixed to a proximal and of the shaft 220. The introducer 200 may be inserted with a dilator inserted therein and through the hemostatic valve associated with the hub 210 of the introducer 200. For example, a rigid dilator may be used that is sized to slide and fit snugly into the lumen extending through the shaft 220. Once the dilator has been removed from the shaft 220, the shaft 201 of the valve repair device 150 can be inserted in the lumen of the sheath 220.

Successful targeting and contacting of the target location on the leaflet 54 can depend at least in part on accurate visualization of the shaft 201 and/or tip/end-effector 204 throughout the process of advancing the tip 204 to the target site. Generally, echocardiographic equipment may be used to provide the necessary or desired intra-operative visualization of the shaft 201 and/or tip 204. For example, for procedures implementing transapical access, rather than intravenous access, in accordance with examples of the present disclosure, fluoroscopy imaging may not provide a practical or tenable solution for imaging. Furthermore, as fluoroscopy can involve exposure of the patient to radiation, such imaging may be undesirable in connection with valve-repair procedures in some situations. Therefore, echo imaging can provide a preferable solution for visibility in valve-repair procedures in accordance with examples of the present disclosure.

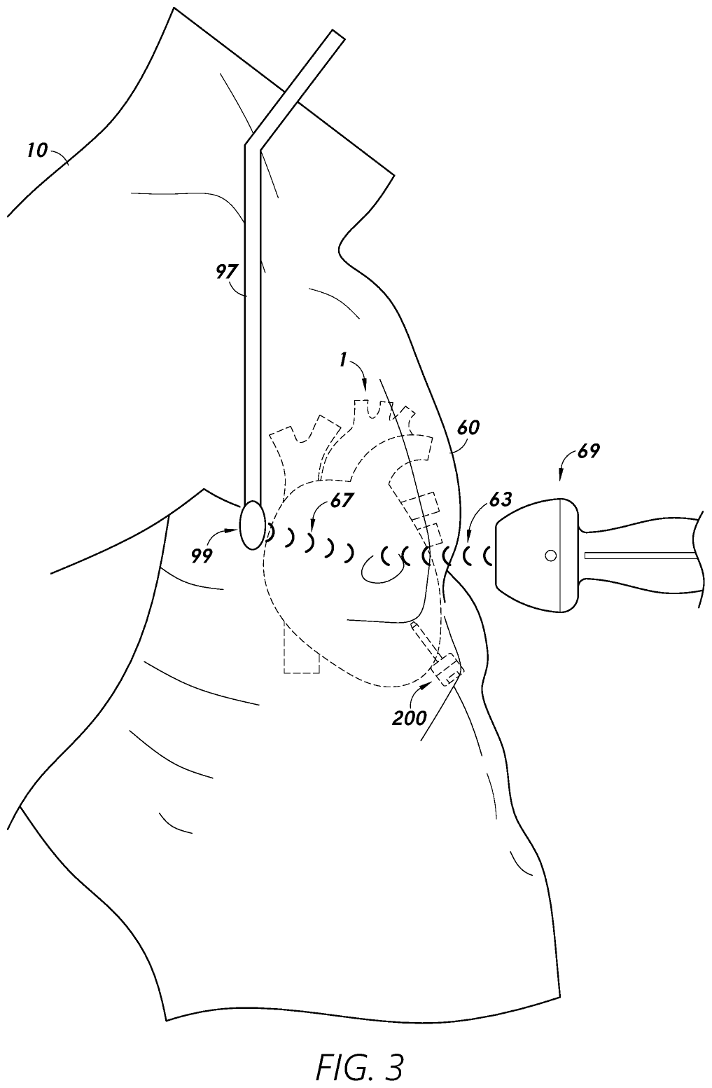

Echo imaging guidance, such as transesophageal echocardiogram (TEE) (2D and/or 3D), transthoracic echocardiogram (TTE), and/or intracardiac echo (ICE), may be used to assist in the advancement and desired positioning of a heart valve repair device shaft and/or an ablation catheter within a heart. FIG. 3 shows a side view of certain imaging devices disposed in positions to generate echo images of cardiac anatomy of a patient in accordance with one or more examples.

Advancement of surgical instruments, including heart valve repair devices and ablation catheters, may be performed in conjunction with echo imaging, direct visualization (e.g., direct transblood visualization), and/or any other suitable remote visualization technique/modality. For example, with respect to the examples of FIGS. 1 and 2, the ablation catheter 106 and/or repair device 250 may be advanced in conjunction with transesophageal (TEE) guidance and/or intracardiac echocardiography (ICE) guidance to facilitate and to direct the movement and proper positioning of the devices. Typical procedures that can be implemented using echo guidance are set forth in Suematsu, Y., J. Thorac. Cardiovasc. Surg. 2005; 130:1348-56, the entire disclosure of which is incorporated herein by reference.

Some examples of the present disclosure relate to devices and methods for providing concurrent port access for working instrument(s) as well as certain imaging devices, such as catheters or other devices configured to generate images using echo or other types of imaging modalities. The terms “echo,” “echo imaging,” “echo image,” “cardiac echo,” “echocardiography,” “echocardiogram,” “medical ultrasound,” “ultrasound,” and like terms are used herein according to their broad and ordinary meanings and may refer to any type of ultrasound (e.g., medical ultrasound), diagnostic sonography, ultrasonography, or any other type of sonic/sound-based imaging technology or modality and/or image(s) generated in connection therewith, whether related to images of cardiac anatomy or other anatomy or subject matter.

In accordance with certain cardiac procedures, such as cardiac tissue ablation and/or heart valve repair procedures, one or more working instruments (e.g., shafts, catheters, etc.) can be passed through a transapical introducer device 200 and advanced inside the ventricle under echo (e.g., ultrasound) guidance. Such imaging implementation may utilize, for example, transthoracic echocardiography (TTE) probe devices or transesophageal echocardiography (TEE) probe devices. However, it should be understood that inventive features disclosed herein may be applicable to any type of imaging devices/modalities.

Echocardiography utilizes sonic imaging to provide sonogram images of the heart, or portion thereof. Generally, echocardiography can utilize two-dimensional (2D), three-dimensional (3D), and/or Doppler ultrasound to create images of the heart, including real-time images. For example, 3D echocardiography technology may implement a matrix array ultrasound probe in combination with appropriate processing system. Such imaging modality may enable detailed anatomical assessment of cardiac pathology, including valvular defects and cardiomyopathies. Real-time 3D echocardiography can be used to guide the location of an ablation catheter or heart valve repair device shaft, as described in detail herein, within a chamber of the heart, such as the left ventricle and/or atrium. Therefore, echo imaging may be used to provide intraoperative assessment of cardiac anatomy and repair system component(s). In some implementations, echocardiography can involve the use ultrasonic pulse echo imaging, or other technology.

FIG. 3 shows a side view of a TIE imaging probe 69 disposed adjacent to a chest wall 60 of a patient in accordance with one or more examples. The echo imaging transducer or probe 69 may be placed on the chest wall 60 of the patient, wherein images are taken through the chest wall 60. Such echocardiography may be referred to as transthoracic echocardiography. Systems like that shown in FIG. 3 may advantageously provide a noninvasive, real-time imaging of the heart 10 during a valve-repair procedure as described herein. The probe 69 can be configured to emit sound waves 61 that generally propagate orthogonally (e.g., at a right angle) with respect to a surface plane of the probe 69. Reflected sound waves are detected by the transducer of the probe 69 and provide imaging information.

FIG. 3 further shows a side view of a transesophageal echocardiography (TEE) imaging probe 99 implemented to generate echo image. The TEE implementation shown in FIG. 3 represents a relatively more invasive form cardiac ultrasound/echo, wherein the ultrasound transducer/probe 99 is placed down the throat of the patient and into the esophagus. Image generation from a position within the patient's esophagus may provide a relatively more detailed, clearer imaging of the heart 1 and any working instruments used in connection with the introducer device 200 than may be typical using transthoracic echocardiography (TIE) imaging. For example, the position of the probe 99 may allow for clearer imaging of certain soft tissue that is not visible in some TTE implementations.

With respect to imaging guidance for ablation catheter procedures, and in particular with respect to accessing the left atrium and locating the pulmonary veins, transesophageal echocardiography (TEE) may be considered sub optimal. Intracardiac echocardiography (ICE) can be used to guide certain percutaneous interventions as an alternative to TTE or TEE. ICE is specialized form of echocardiography that generally involves the use of catheters to insert an ultrasound probe inside the heart or other anatomy to view structures from within. Different types of ICE technologies can be implementing, including radial or rotational ICE, which involves the use of a piezoelectric crystal mounted at the tip of a 6- to 10-French catheter. A rotating transducer can provide cross-sectional images in a 360-degree radial plane perpendicular to the long axis of the catheter. Rotational ICE generally operates at imaging frequencies of 9 to 12 MHz, which is useful for near-field imaging of up to 6 to 8 cm but limited for far-field imaging.

Intracardiac echocardiography can further be implemented as phased-array ICE. In such technologies, a multiple-element (e.g., 64-element) transducer can be mounted on the distal end of an 8- or 10-French steerable catheter that can be deflected in four directions (anterior, posterior, right, and left). Such a device can produce a wedge-shaped image that is displayed on a conventional ultrasound workstation. Phased-array ICE can provide certain advantages over the mechanical rotational systems, including greater depth of penetration (up to 15 cm), greater maneuverability, and/or the ability to acquire Doppler and color flow imaging. Some uses of ICE are described in Saji, M., “Adjunctive Intracardiac Echocardiography Imaging from the Left Ventricle to Guide Percutaneous Mitral Valve Repair With the MitraClip in Patients With Failed Prior Surgical Rings”, Catheterization and Cardiovascular Interventions, 87:E75-E82 (2016), the entire disclosure of which is incorporated herein by reference.

Catheter-based intracardiac echocardiography (ICE) can advantageously allow for imaging inside the heart to visualize cardiac structures and blood flow using Doppler imaging with image quality that is generally comparable to that of transesophageal echocardiography. The use of ICE imaging can provide a relatively clear image and precise, real-time visualization of the both intracardiac anatomy and devices positioned within the heart. In some cases, ICE imaging devices can be used to identify anatomical structures and visualize the position/location of instrumentation/devices present in the imaging field relative to such anatomical structures.

According to some procedures, an ICE catheter probe may be advanced to the inside of the heart through a transcatheter approach through the femoral artery or other arterial or venous blood vessel(s). Furthermore, according to some implementations, an ICE catheter may be used to image the heart from an esophageal access position as an alternative to transesophageal echocardiography as described above. For example, the ICE catheter may generally be substantially smaller in profile compared to the probe 99 shown in FIG. 3, and therefore can be more comfortable for the patient in a conscious sedation procedure. Furthermore, the presence of the smaller ICE catheter may be less likely to induce gagging or other uncomfortable reflex in the patient in a conscious sedation state.

Examples of the present disclosure relate to multi-lumen valved introducer systems and devices that can be used in minimally invasive beating-heart mitral valve repair procedures and/or other cardiac or medical procedures. For example, multi-lumen introducer devices of the present disclosure can be configured to receive two or more catheter-type instruments, wherein the introducer device is used as a single-port access to the left ventricle or other chamber of the heart. With respect to the mitral valve repair procedures as described herein, a multi-lumen introducer in accordance with aspects of the present disclosure can facilitate insertion of a tissue anchor delivery system through one channel/lumen of the multi-lumen device, whereas a second channel/lumen of the device may be used to introduce an intracardiac echocardiography (ICE) imaging catheter to the heart to provide imaging for the mitral valve repair procedure.

The use of intracardiac echocardiography as an alternative to, or in conjunction with, transesophageal echocardiography, can advantageously provide enhanced visualization of intracardiac structures, wherein such imaging may guide the navigation of mitral valve repair system components within the ventricle and/or atrium of the heart and/or improve the accuracy of targeting of the target mitral valve leaflet for leaflet repair. ICE catheter imaging during a cardiac ablation or valve repair procedure can be facilitated by use of multi-lumen introducer devices disclosed herein. Multi-lumen introducers of the present disclosure can be used to perform adjunctive surgical atrial fibrillation ablation through transapical access through the left ventricle in connection with an off-pump beating heart mitral valve surgery, wherein separate ports and lumens of the introducer device may be used for access to the ventricle and/or atrium using an ICE catheter and an ablation catheter, respectively. That is, such procedure may be implemented through the left ventricle, wherein an ablation catheter is passed through one channel/lumen of the introducer, while another channel/lumen is used for intracardiac echocardiography.

The percutaneous portal access solutions using multi-lumen introducer devices presented herein can provide enhanced and/or improved means or techniques for implementing mitral valve repair surgery, as well as for other minimally-invasive surgical procedures that are conducive to introducer portal access. As described above, it can be desirable to treat atrial fibrillation in connection with mitral valve repair or replacement procedures. However, when a single-lumen introducer as shown in FIG. 2 is implemented for leaflet tethering in connection with a valve repair device 250 as shown in FIG. 2, it may not be tenable to treat atrial fibrillation in a single setting with performance of the mitral valve repair procedure. Therefore, it may be necessary to bring the patient back for atrial fibrillation treatment using ablation or the like, which may require engagement of an electrophysiology specialist for the purpose of implementing endocardium ablation through a transcatheter approach. Such procedures can be undesirable due to the additional patient admission required, such as with respect to exposure of the patient to hospital-acquired infection and/or other complications associated with additional admission for treatment. Furthermore, during the time between the valve (e.g., mitral valve) repair procedure and the ablation procedure, the patient may suffer from certain exacerbations and/or complications associated with atrial fibrillation, including the possibility of stroke or other serious complications. Furthermore, transcatheter procedures may generally be reserved for patients considered to be probative-risk or high-risk for surgery. Therefore, patients with atrial fibrillation who are not considered probative-risk or high-risk patients may not be candidates for transcatheter ablation procedures. Therefore, where mitral valve repair is implemented with respect to patients who are not probative-risk for high-risk patients, subsequent transcatheter ablation procedures may not be practical.

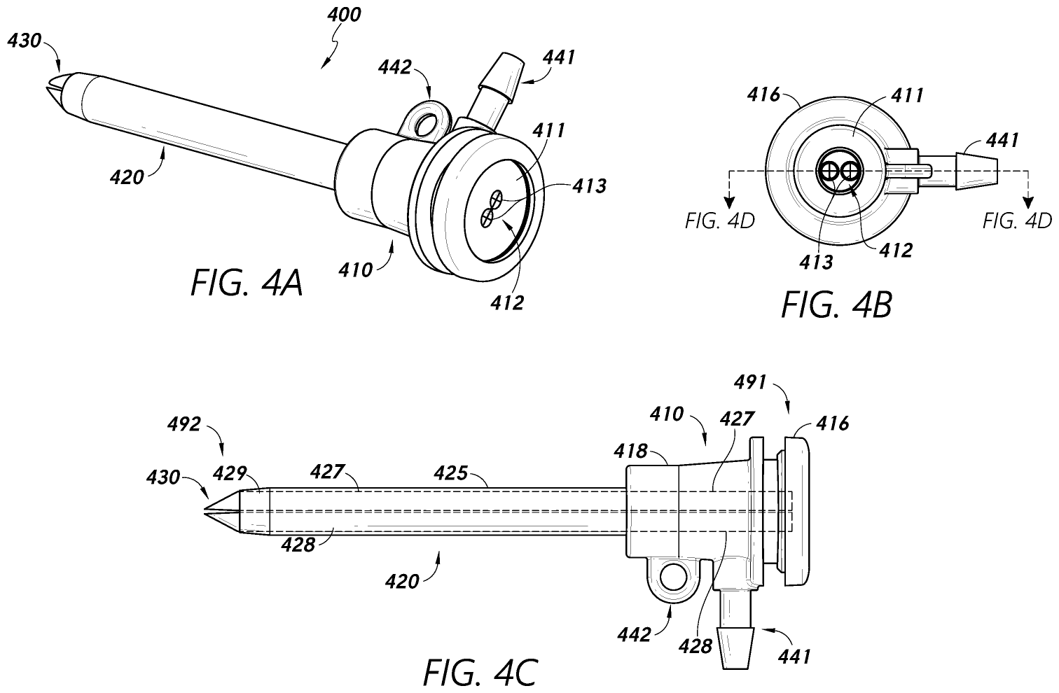

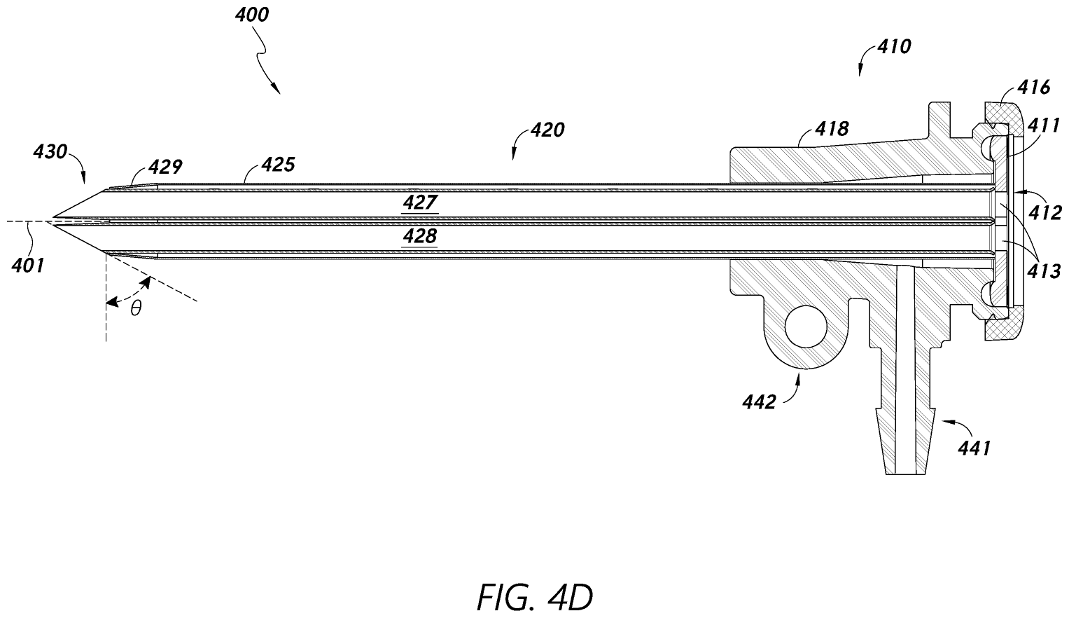

FIGS. 4A-4D show isometric, proximal end, side, and cross-sectional side views, respectively, of a multi-lumen introducer device 400 in accordance with one or more examples. The multi-lumen introducer 400 can include a proximal hub 410 and a tubular body portion 420. Two or more channels or lumens 427, 428 may run lengthwise within at least a portion of the introducer device 400 from a proximal end portion 491 to a distal end portion 492 thereof. The elongate portion 420 of the sheath housing the plurality of channel/lumens 427, 428 of the device 400 can have any cross-sectional shape or area. For example, the sheath 420 can have a circular or ovoid cross-sectional shape.

Each of the lumens 427, 428 provides a port 412 of access through the introducer device 400 for working instrument(s) disposed therein. Each of the lumens 427, 428, at or near a proximal portion thereof, is associated with a respective valve 413. Such valves may advantageously be leak-resistant elastomeric valves, or the like, and may be configured to allow certain instruments to pass therethrough and form a seal around such instruments. The valves 413 can further be configured to close and form a reliable hemostatic seal in the absence of elongate instruments being disposed within the lumens 427, 428, as shown in FIGS. 4A and 4B. In some examples, each of the ports 412 has an independent hemostatic valve 413 at or near a proximal opening thereof.

In some examples, a dilator may be inserted through one of the channels 427, 428 and used to facilitate insertion of the introducer 400 into the target anatomy, wherein, after placement of the introducer device 400 in the target anatomy, the dilator may be removed to allow for reuse of the channel/lumen for another instrument, such as an ablation catheter, valve repair shaft, imaging catheter, or the like. Although examples of the present disclosure describe utilizing lumens of a multi-lumen introducer device for ablation catheters, valve repair device shafts, dilators, and imaging catheters, it should be understood that such lumens/ports can be utilized for other types of medical instruments, including grasping devices, suturing and/or suture-deployment devices, cameras, or the like.

In some examples, the sheath portion 420 comprises a tube having a plurality of lumens formed therein. For example, the sheath 420 may comprise an extruded tube produced using an extrusion device/system, wherein such extrusion implements a plurality of lengthwise fluidly-isolated channels/lumens. In some examples, the sheath portion 420 comprises an outer tube 425 having a plurality of inner tubes disposed therein, which may have their own tube walls, as shown in FIG. 4D. In such cases, certain space/gaps may exist between portion of the inner tubes and the outer tube 425. Multi-lumen introducer devices in accordance with aspects of the present disclosure, such as the multi-lumen introducer 400, can advantageously reduce the amount of risk presented in connection with cardiac procedures by utilizing a single transapical access point rather than separate access point/tubes for multiple-instrument operation.

In some examples, the distal end 430 of the sheath portion 420 of the multi-lumen introducer device 400 may be tapered, as shown. For example, the distal end 430 is shown having a taper of some angle θ, which may be any acute angle. The taper may be primarily with respect to the lumens/channels 427, 428. That is, the distal end portions of the lumens/channels 427, 428 may form the tapered shape of the distal end 430 of the sheath 420, as shown. The taper of the distal end 430 of the introducer 400 can facilitate advancement thereof into the target anatomy.

As shown in the examples of FIGS. 4A-4D, in some implementations, a multi-lumen introducer device may include two or more lumens/channels that are each tapered towards an axial center 401 of the sheath portion 420 of the introducer 400. For example, with respect to the example of FIGS. 4A-4D, the lumens/channels 427, 428 at distal ends thereof may each be tapered towards the axial center 401 of the sheath portion 420. That is, each lumen of a multi-lumen introducer device may be tapered at an angle and/or direction that varies from a corresponding taper of another lumen/channel of the introducer. With the opposing tapers of the lumens/channels 427, 428, the inside portions of such lumens/channels with respect to the axis 401 of the sheath portion 420 may together form a point or apex that may facilitate driving of the sheath 420 through the anatomical tissue of the patient to introduce the distal end 430 in the target anatomy.

In some examples, each of a plurality of lumens/channels of a multi-lumen introducer device may be tapered from an outer portion thereof to an inner portion thereof, wherein the outer portion corresponds to a portion of the lumen/channel that is radially farthest outward with respect to the axis of the sheath of the introducer, whereas the inner portion corresponds to a portion of the lumen/channel that is radially closest to the axis of the sheath. Although certain description herein describes distal ends of lumens/channels as being tapered, it should be understood that the tapered shape/form of a sheath portion of a multi-lumen introducer device in accordance with aspects of the present disclosure may include certain tapering as described herein, where in such tapered surfaces/forms may be associated with the lumens/channels of the sheath and/or other physical structure/form of the sheath that is not part of a lumen or channel.

As with other multi-lumen introducer devices of the present disclosure, the introducer device 400 can provide a single access site for simultaneously introducing two or more catheters into the left ventricle or other anatomical site through the first 427 and second 428 lumens, respectively. That is, the multi-lumen introducer device 400 of the can be configured to receive two or more catheter instruments, including an intracardiac echocardiography catheter that is used concurrently alongside a working catheter instrument, such as an ablation catheter. In some implementations, the multi-lumen introducer device 400 can be used to receive a mitral valve repair device shaft in place of, or in addition to, an ablation catheter. Through the use of the plurality of lumens of the multi-lumen introducer 400 device for imaging, valve repair, and/or ablation, the introducer site may remain substantially intact and secured to the left ventricle wall or other anatomy throughout a valve repair and/or ablation procedure.

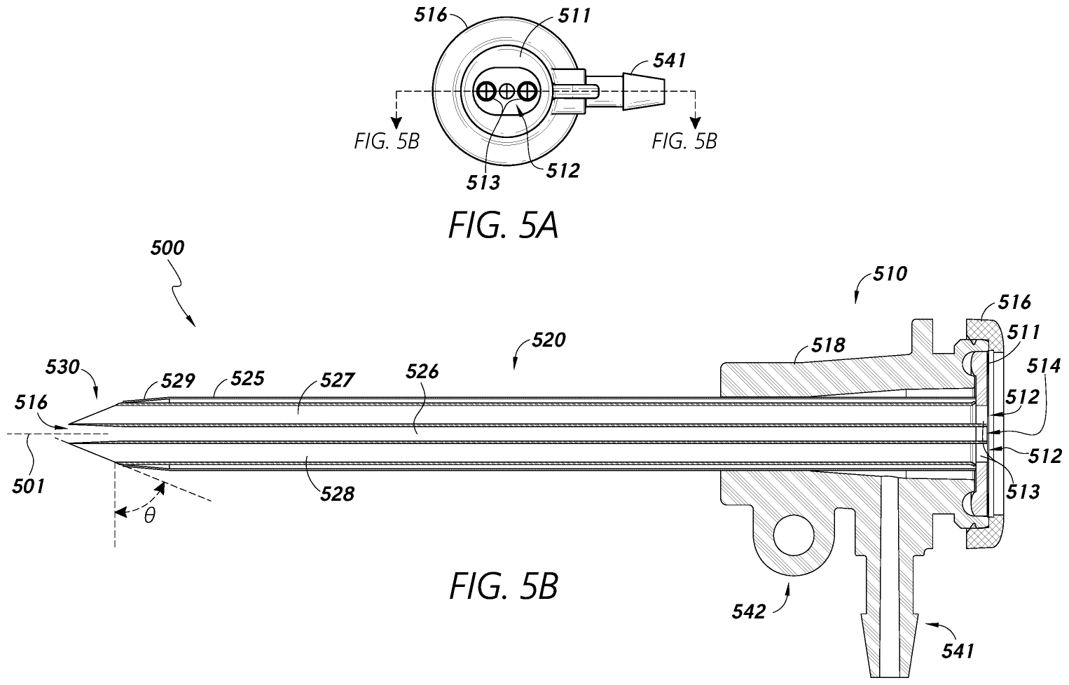

FIGS. 5A and 5B show proximal end and cross-sectional side views, respectively, of a multi-lumen introducer device 500 in accordance with one or more examples of the present disclosure. In some examples, multi-lumen introducer devices of the present disclosure include a centrally-located lumen configured to receive a dilator or other device or instrument. For example, as with the introducer device 500 of FIGS. 5A and 5B, a centrally-positioned port 514 configured to receive a dilator therethrough can be flanked on one or more sides by adjacent peripherally-located ports 512 that open to tubular channels 527, 528 that are configured to receive relatively small-diameter catheters having flexible or rigid shafts, such as intracardiac radiography catheters, valve repair device shafts, and/or ablation catheters, as described in detail herein.

The inclusion of a central dilator port 514 can facilitate installation/insertion of the introducer device 500 by allowing for a dilator to project from a distal end 516 thereof and facilitate/promote the widening of the access opening in the anatomy of the chest, heart, and/or intervening tissue leading to the left (or right) ventricle access. For example, the heart may initially be punctured with a needle, wherein a guidewire may be inserted through the needle puncture access, after which a dilator may be used to guide the introducer into the heart. In some implementations, one or more instruments may be inserted through the instrument lumens 527, 528 while the dilator (not shown) is present in the dilator channel 526, wherein the dilator may be removed at any point after insertion of the introducer 500.

In some examples, the introducer 500 includes separate, fluidly-isolated lumens 527, 528 corresponding to the outer instrument ports 512, whereas the central dilator port 514 may or may not have a dedicated lumen structure or form (e.g., tube structure) associated therewith. For example, in some instances, the space within the sheath portion 520 may include the fluidly-isolated lumens 527, 528, which may be formed by one or more shafts or other tube-type structures, whereas the remaining space within the outer tube/sheath 520 may be generally open and/or provide a cavity/channel through which a dilator may be advanced and/or disposed.

Although working instrument and dilator lumens/channels are shown in FIGS. 5A and 5B having certain relative sizes, it should be understood that such lumens/channels may have any suitable or desirable sizes and/or configurations. For example, in some instances, the dilator lumen/channel 526 may have a greater cross-sectional area and/or diameter than the outer lumens/channels 527, 528. Alternatively, in some examples, the dilator lumen/channel 526 may have a smaller cross-sectional area and/or diameter than the outer working instrument lumens/channels 527, 528. Furthermore, although the dilator lumen/channel 526 is described as being disposed generally at an axial center of the sheath portion 520, in some implementations, the dilator lumen/channel 526 may be offset from the axial center of the sheath 520. For example, one or more working instrument lumens/channels may be positioned generally on or about the central axis 501.

As with other multi-lumen introducer devices disclosed herein, the distal end 530 of the sheath 520 may be tapered toward an axial center 501 of the sheath 520 to facilitate insertion of the introducer device 500 in the patient anatomy. For example, in some instances, outer working lumens/channels 527, 528 may each be tapered from an outside portion thereof to an inside portion thereof, wherein the outer portions correspond to portions of the lumens/channels associated with a radially outward-most portion thereof, whereas inner portions correspond to portions of the lumens/channels associated with radially inward-most portion thereof, as shown in FIG. 5B. In instances in which the dilator channel/lumen 526 is positioned generally on or about the axis 501 of the sheath 520, the tapering of the working channels/lumens 527, 528 may not come together to a point in the manner as shown in FIG. 4D, but rather a gap/opening 516 may be present between the working lumens/channels 527, 528 that is associated with the dilator lumen/channel 526.

As with other multi-lumen introducer devices of the present disclosure, the introducer device 500 can provide a single access site for simultaneously introducing two or more catheters into the left ventricle or other anatomical site through the first 527 and second 528 lumens, respectively. That is, the multi-lumen introducer device 500 of the can be configured to receive two or more catheter instruments, including an intracardiac echocardiography catheter that is used concurrently alongside a working catheter instrument, such as an ablation catheter. In some implementations, the multi-lumen introducer device 500 can be used to receive a mitral valve repair device shaft in place of, or in addition to, an ablation catheter. Through the use of the plurality of lumens of the multi-lumen introducer 500 device for imaging, valve repair, and/or ablation, the introducer site may remain substantially intact and secured to the left ventricle wall or other anatomy throughout a valve repair and/or ablation procedure.

According to some aspects, examples of multi-lumen introducer devices, such as those shown in FIGS. 4 and 5 and described above, can facilitate minimally-invasive approaches for catheter ablation procedures to treat atrial fibrillation on a beating heart. For example, a multi-lumen introducer device in accordance with aspects of the present disclosure can be used to introduce two or more catheter devices into and/or through the left ventricle of the heart. Such catheters may comprise one or more atrial ablation catheters and/or intracardiac echocardiography catheters. For example, the atrial ablation catheters may be implemented as radiofrequency ablation devices and/or cryoablation devices, as described in detail above. However, it should be understood that any type of catheter ablation device may be implemented in connection with examples of the present disclosure.

With respect to the use of intracardiac echocardiography catheters in multi-lumen introducer devices of the present disclosure, such imaging catheters may be implemented as a tool to guide and position a medical instrument, such as an ablation catheter or heart valve repair device shaft as referenced above, in the left ventricle and/or atrium to provide real-time, high-resolution images of intracardiac anatomical structures and assist in visualizing the location, position, and/or orientation of such instrument relative to such anatomical structures.

In some implementations, surgical mitral valve repair may be implemented through access via a multi-lumen introducer device after atrial ablation has been performed via the introducer device with the utilization of intracardiac echocardiography imaging, wherein one or more lumens of the introducer device may be utilized for imaging to guide the valve repair device/shaft. The use of intracardiac echocardiography with a multi-lumen introducer device, wherein an imaging catheter accesses the ventricle the ventricle of a heart through a channel/lumen of the multi-lumen introducer device, can provide improved and/or enhanced views of cardiac structures and/or reduce risks by avoiding entanglement with native chordae tendinea during maneuvering and navigation in the left ventricle, as well as aiding accurate targeting of the mitral valve leaflet. By using intracardiac echocardiography catheters through transapical access ports/lumens of a multi-lumen introducer device in accordance with aspects of the present disclosure, the operating surgeon may be able to control the imaging device(s), rather than requiring the assistance of a sonographer. Although particular imaging and ablation features are described with respect to particular examples disclosed, it should be understood that such features can be combined with, or substituted with, any other type of imaging, ablation, valve repair, or other type of instrumentation.

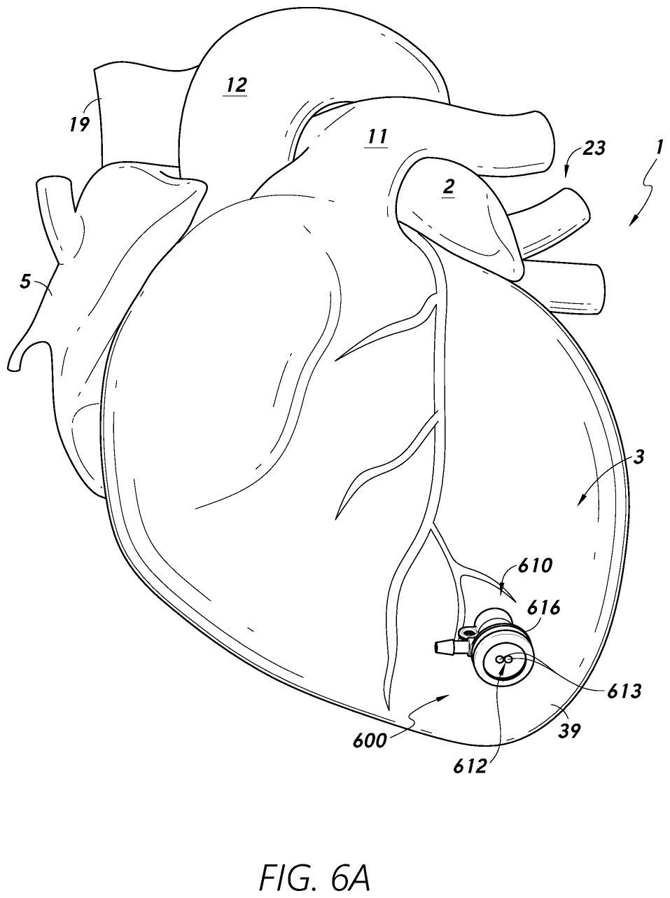

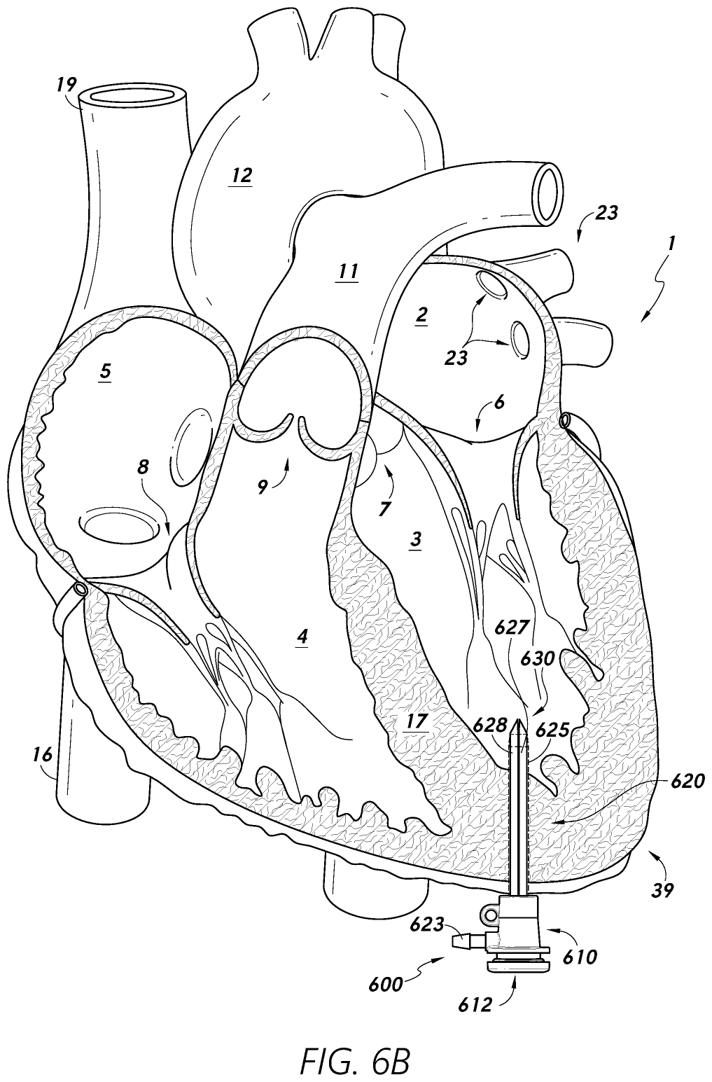

FIGS. 6A and 6B shows perspective and cutaway views, respectively, of a heart 1 having a multi-lumen introducer device 600 disposed at least partially therein in accordance with one or more examples. The transapical insertion of the introducer device 600 can provide a relatively short-length, direct access to the interior of the heart 1 through which multiple instruments may access the relevant cardiac anatomy in connection with a single operation/sitting, thereby facilitating atrial tissue ablation for atrial fibrillation treatment in connection with valve repair or other procedure(s). The multi-lumen introducer 600 includes a plurality of fluidly-isolated ports 612, each associated with a separate access lumen and hemostatic valve 613.

The hemostatic introducer 600 may be inserted into the target ventricle at a tip 630 associated with distal ends of a plurality of lumens/channels 627, 628 of the introducer 600. The sheath portion 620 of the introducer 600 may be used to span the distance between the outside of the heart 1 and the inside of the ventricle 3 to provide access through the intervening tissue/space. The body/hub 610 of the introducer 600 may be used to secure the introducer 600 to the pericardium of the heart for stable entry of working instruments through the lumens of the sheath 620 and/or to control the amount of bleed-back during the valve-repair procedure. In some instances, a female Luer may be used to de-air the introducer 600 through a port 623 prior to use and/or to connect a fluid flush, such as a heparin flush, during a valve-repair procedure. A dilator (not shown) may be used to guide the introducer into the target ventricle. For example, the dilator may be used to guide the introducer 600 into the left ventricle off-apex, as described in detail herein, and may be placed through/within one of the lumens of the multi-lumen introducer 600. In some implementations, a tie-down eyelet is used to secure the introducer 600 during the relevant cardiac procedure.

The sheath portion 620 of the introducer 600 provides a housing for the lumens/channels 627, 628, which provide respective fluidly-isolated conduits into a target surgical area or chamber, such as a ventricle of a heart. In some instances, the introducer 600 comprises one or more hemostasis valves associated with each of a plurality of channels/lumens 627, 628 and/or ports 612. Such hemostasis valve(s) may comprise silicone or other flexible material configured to keep blood from flowing out of the respective port 612. The ports 612 may serve as working instrument (e.g., catheter/shaft) insertion ports, wherein inserted instruments may pass through lumens/channels associated with the respective ports 612 of the introducer 600 and out the distal end 630 of the sheath 620 into the target chamber 3. One or more of the ports 612 may further be dimensioned to accommodate insertion of dilator devices used to guide the introducer 600 into the target chamber (e.g., left ventricle, off-apex). The distal end 630 of the introducer 600 may have a tapered shape to seal against the inserted instruments/devices and/or to facilitate insertion of the sheath 620 through biological tissue.

The introducer may include an outer tube 625 that surrounds the lumens/channels 627, 628, wherein the lumens/channels 627, 628 can include separate tube structures, or may be formed in a common form/structure of the sheath portion 620. In some implementations, the introducer 600 includes a lumen/channel for insertion of a dilator device, wherein such lumen/channel may be one of the lumens/channels 627, 628, or may be a separate lumen/channel. For example, the introducer 600 may include a central dilator channel that is similar in one or more respects to the dilator channel 526 shown in FIG. 5 and described above.

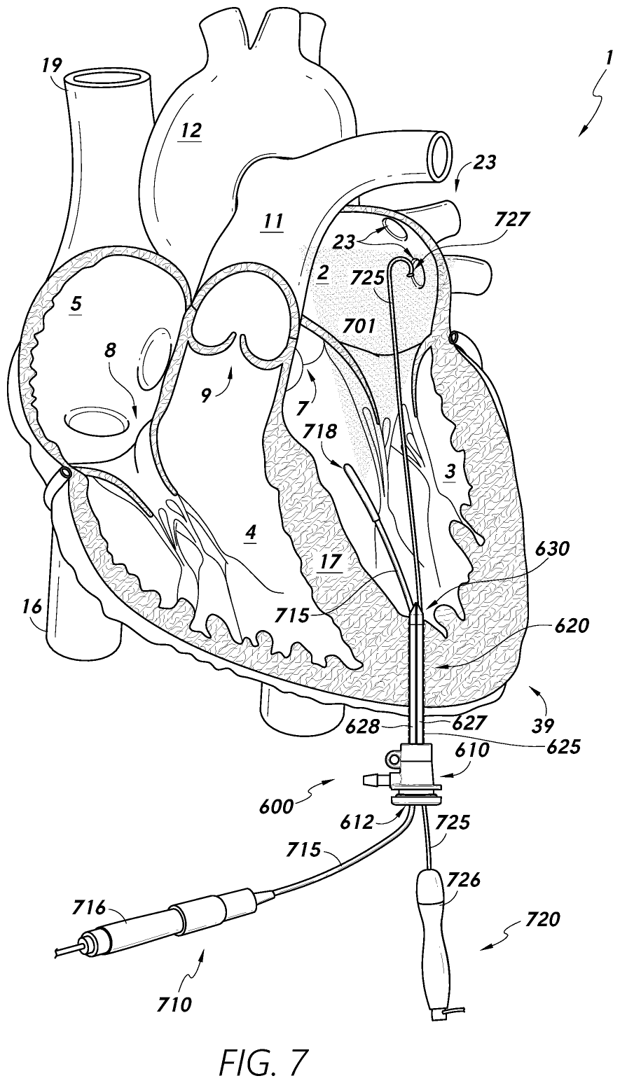

FIG. 7 is a cutaway view of the heart 1 and multi-lumen introducer 600 shown in FIGS. 6A and 6B with a plurality of medical instruments 715, 725 accessing chambers of the heart 1 through respective lumens 628, 627 of the multi-lumen introducer device. The introducer device 600 can be configured to allow two catheter instruments through a single access structure provided by the sheath component 620, which can pierce through the relevant biological tissue, such as the wall of the ventricle. The introducer 600 may advantageously be used for operations in which the heart continues to beat, as opposed to bypass surgical operations.

The device 600 may facilitate transapical access for atrial ablation using an ablation catheter device 720 to treat atrial fibrillation concurrently and/or in conjunction with a minimally invasive valve repair. For example, in some implementations, an imaging catheter device 710, which may comprise a handle portion 716, a catheter portion 715, and/or a distal imaging element 718, can be inserted in one channel 628 of the multi-lumen introducer 600, wherein the imaging catheter 710 may be configured to generate images from within the heart/ventricle. The ablation catheter 720 may be inserted concurrently with the imaging device 710 being disposed in the ventricle. The intracardiac echocardiography imaging provided by the imaging device 710 can provide real-time imaging of the atrial structures (e.g., pulmonary vein(s)) 23 around which ablation may be performed. With enhanced visualization, the ablation catheter 720 can be positioned relatively precisely in the areas in need of ablation. Once ablation is complete, the ablation catheter can be switched with a valve repair shaft/device, or other instrument, all while keeping the multi-lumen introducer 600 and/or imaging catheter 715 in place, such that leaflet repair with chordal implantation, for example, may be performed while the imaging catheter 715 remains inside the heart. Execution of tissue ablation using the ablation catheter 720 may be guided at least in part by multi-lead EKG signals that are configured to monitor electrical signals in the heart and guide the physician to areas of problematic electrical signals.

Imaging access through a transapical introducer access as shown in FIG. 7 can be preferable over esophageal imaging access due to comfort and/or proximity considerations. As described above, the ablation catheter 725 may be used to emit radiofrequency energy at a distal end 727 thereof to create circular or otherwise patterned scars around one or more of the pulmonary veins 23 or other anatomy. In some examples, the ablation catheter 725 is configured to ablate using cryogenic mechanism(s), as described above. For example, the catheter 725 may have a distal balloon feature that is filled at least in part with a substance configured to freeze cardiac tissue that comes in contact therewith to cause scarring of the tissue.

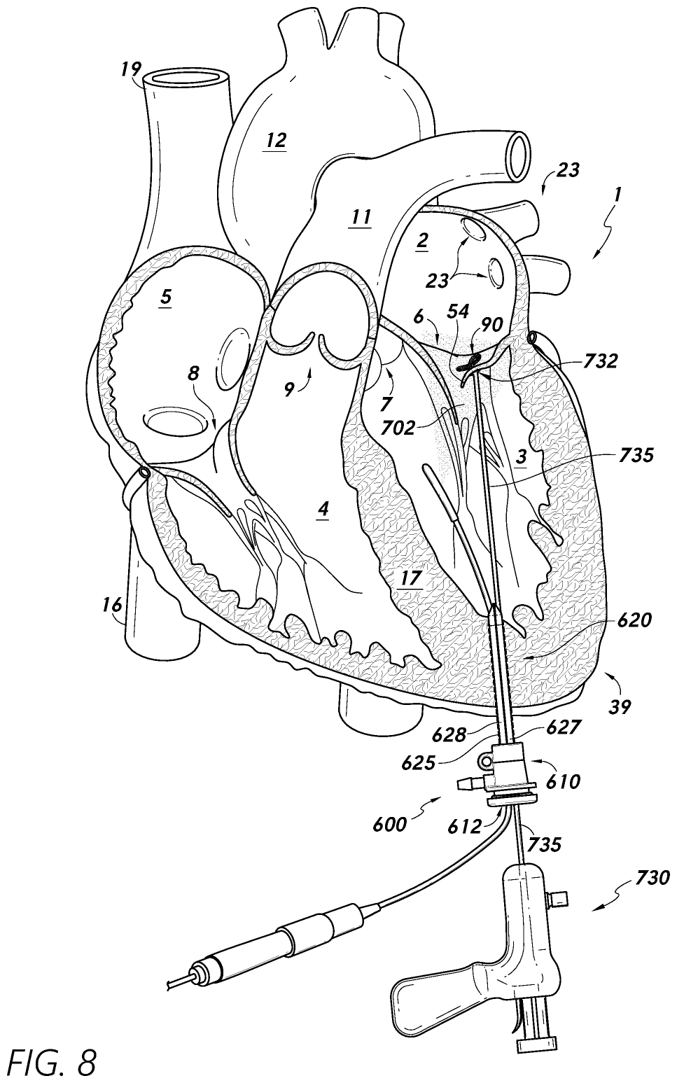

FIG. 8 is a cutaway view of the heart 1 and multi-lumen introducer 600 shown in FIGS. 6A and 6B with a plurality of medical instruments accessing chambers of the heart through respective lumens of the multi-lumen introducer device. The introducer 600 may allow for the implementation of a valve leaflet tethering procedure using a heart valve repair device 730 guided by intracardiac echocardiography using the imaging catheter device 710. For example, the multiple ports 612 of the introducer 600 can allow for the insertion of the intracardiac echocardiography catheter 715 through one lumen/channel of the introducer 600, while the other lumen/channel may be reserved for the shaft 735 of the heart valve repair device 730. Intracardiac imaging may improve imaging of the cardiac structures, provide enhanced visualization, augment guidance visualization for the left ventricle, and/or improve the accuracy in targeting of a target valve leaflet 54.

The shaft 735 may represent a relatively low-profile delivery device, which may be dimensioned to fit within the lumen 627 of the introducer 600. For example, the shaft 735 may be an about 3 mm (9 Fr) shaft. Furthermore, the tip (e.g., end effector) 732 may advantageously be flexible to allow for insertion into the lumen 627 even where the lumen 627 has a smaller diameter than the extended diameter of the tip 732.

Multi-lumen introducer devices disclosed herein can enhance surgical procedures by improving safety and precision, shortening surgical time, reducing risks and post-operative complications, and/or improving surgical outcomes. Although disclosed in connection with transapical cardiac access procedures, it should be understood that multi-lumen introducer devices in accordance with aspects of the present disclosure may be utilized in connection with any minimally invasive surgery that is conducive to portal access.

In some implementations, the heart valve repair device 730 can be used to deploy one or more tissue anchors 90, such as on an atrial side of the target valve leaflet 54. For example, the tissue anchor 90 may be deployed in the leaflet 54 using the repair device 730, after which the shaft 735 of the repair device 730 may be withdrawn from the lumen 627 to allow for insertion therein of an ablation catheter as shown in FIG. 7, wherein the imaging catheter 715 can remain disposed in and through the lumen 628 of the introducer 600 throughout the transition between the valve repair device 730 and the ablation catheter device.

FIGS. 7 and 8 show example combinations of instrumentation that may be used concurrently using a multi-lumen introducer according to aspects of the present disclosure. However, it should be understood that other combinations of instruments may be implemented with a multi-lumen introducer as disclosed herein, including, for example, grasping devices/instruments, edge-to-edge leaflet attachment devices, papillary muscle adjustment devices and systems, camera systems, fluid irrigation and/or aspiration devices (e.g., irrigation catheter used to maintain translucency for a camera scope device), and/or the like.

FIGS. 9-1 through 9-5 provide a flow diagram illustrating a process 800 for performing a medical procedure in accordance with one or more examples. FIGS. 10-1 through 10-5 provide cross-sectional images of cardiac anatomy and certain devices/systems corresponding to operations of the process of FIGS. 9-1 through 9-5 according to one or more examples of the present disclosure. The process 800 may relate to the insertion/placement and use of a hemostatic multi-lumen introducer device transapically in the left ventricle to provide an access point through the ventricular wall and into the left ventricle.

At block 802, the process 800 involves accessing the apex of the heart of a patient through the chest wall in a minimally invasive manner. For example, such access may be made between ribs of the patient, or beneath the rib cage. At block 804, the process 800 involves puncturing the ventricle wall with a needle device 80, which may include a handle 81 and a pointed distal tip portion 82, which may be advanced through the tissue of the ventricle wall and into the ventricle 3. As an example implementation, once a suitable entry point has been established, the surgeon can use one or more sutures to make a series of stiches in one or more concentric circles in the myocardium at the desired location to create a “purse-string” closure. The Seldinger technique can be used to access the left ventricle in the area surrounded by the purse-string suture by puncturing the myocardium using the needle device 82, which may comprise a small sharp hollow needle (a “trocar”).

At block 806, the process 800 involves inserting a guidewire 85 through a lumen of the needle 82 to provide access into the left ventricle 3 for the guidewire 85. For example, the guidewire 85 may be directed through a proximal port or opening in the needle device 80 and may pass through a central lumen within the center of the elongate needle 82. At block 808, the process 800 involves removing the needle device 80 while maintaining the guidewire 85 in place please partially within the ventricle 30. For example, the needle device 80 may be withdrawn proximally over the proximal end of the guidewire 85.

At block 810, the process 800 involves advancing a dilator 86 over the guidewire 85 to dilate/expand the opening in the tissue wall 18 through which the guidewire 85 and needle 80 have been passed. The dilator 86 may serve to expand the opening/pathway to a degree to allow for insertion of the introducer device. In some implementations, different sized dilators may be sequentially inserted to gradually dilate the access pathway incrementally to prevent tissue damage and to match the dilation to the size of the introducer sheath to be inserted.

At block 811, the process 800 involves removing the dilator 86 to allow for insertion of the introducer device 91. At block 812, the process 800 involves inserting a multi-lumen introducer device 91, which may have characteristics as described in connection with various examples disclosed herein, through the dilated access path through the tissue wall 18 to the ventricle 3. That is, insertion of the introducer 91 may involve advancing a sheath component 92 thereof such that a distal end 93 of the sheath 92 penetrates and enters the ventricle 3, as shown in image 913. Although removal of the dilator 86 is described as occurring prior to insertion of the multi-lumen introducer 91, in some examples, the introducer 91 may be inserted by distally advancing the introducer sheath 92 over the dilator 87.

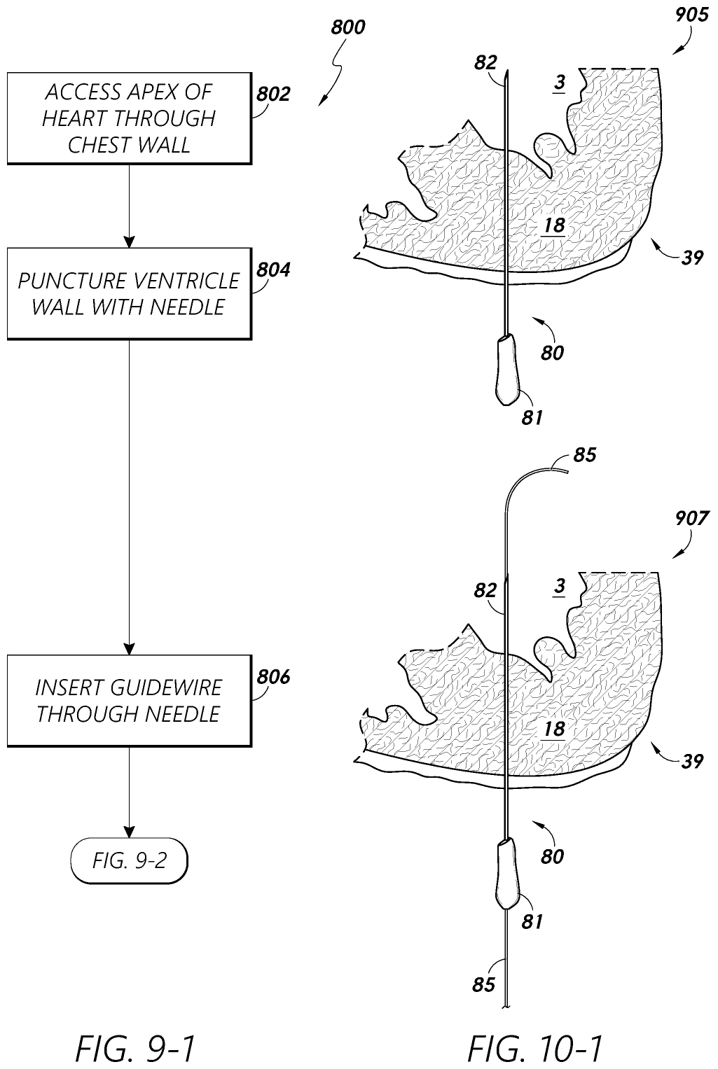

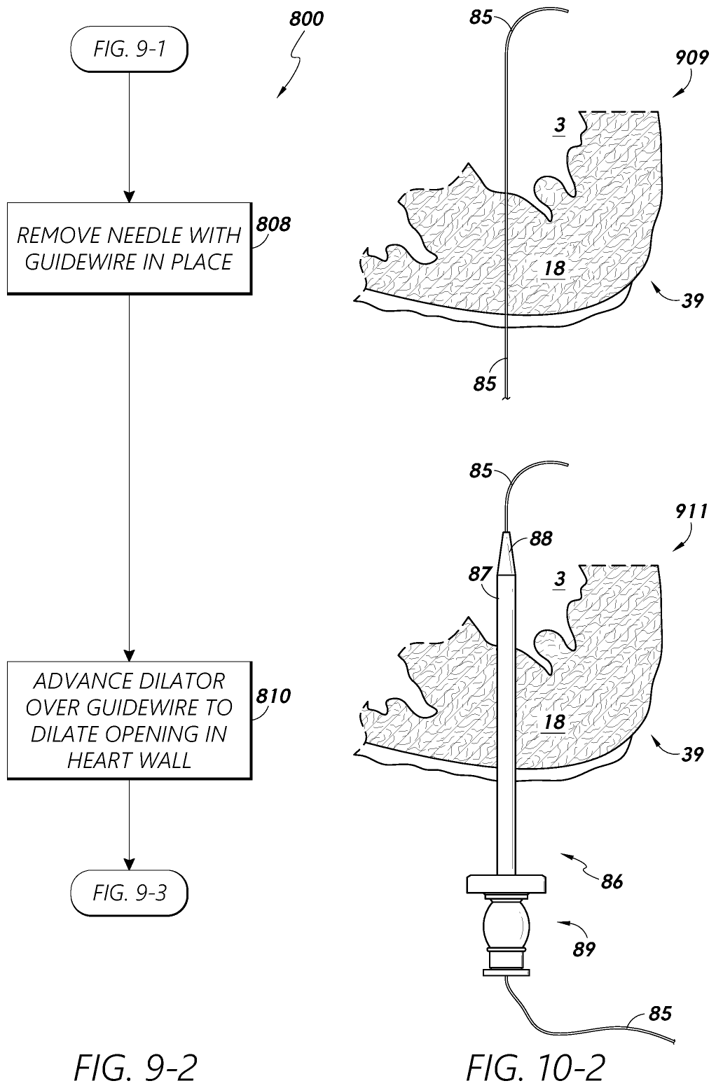

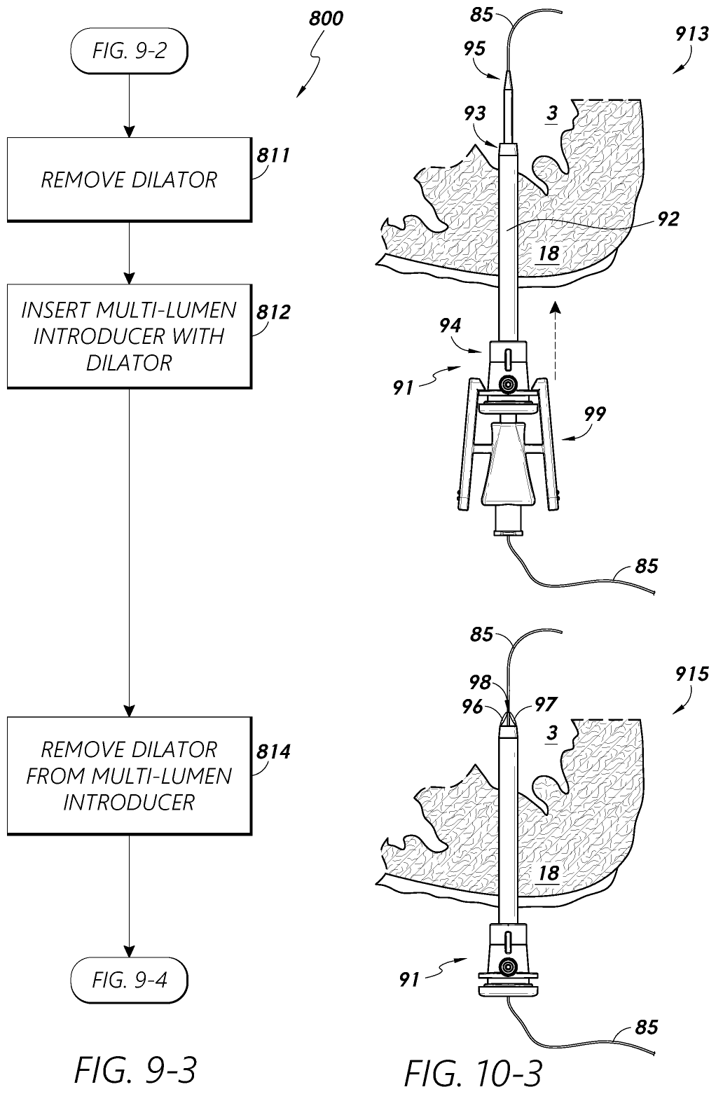

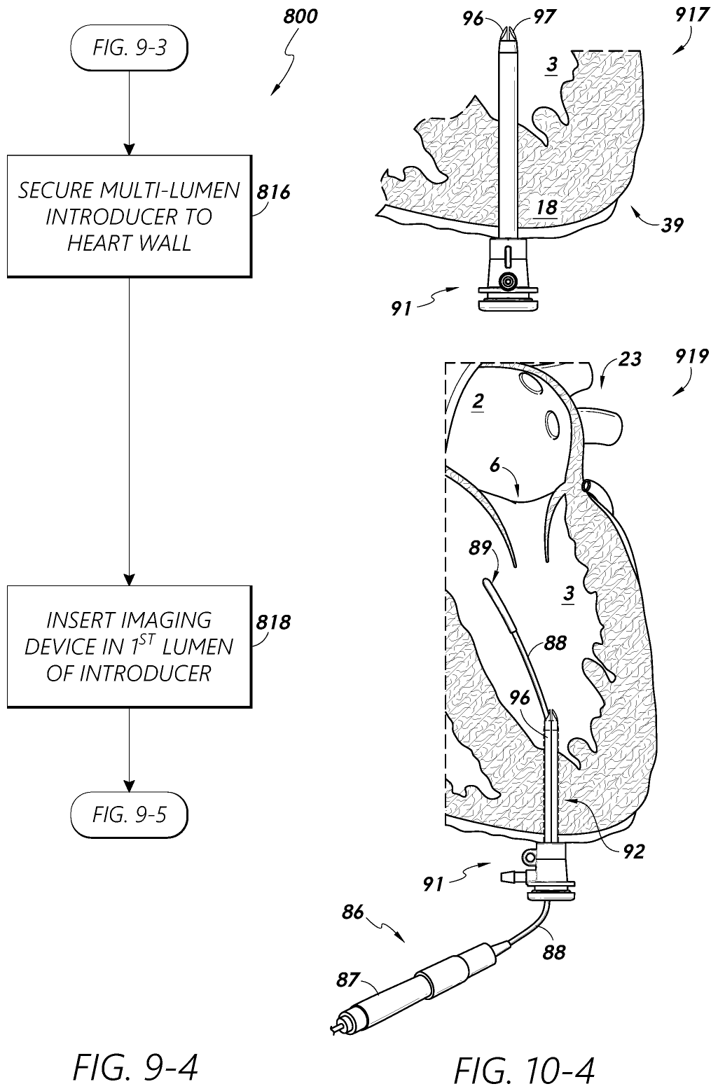

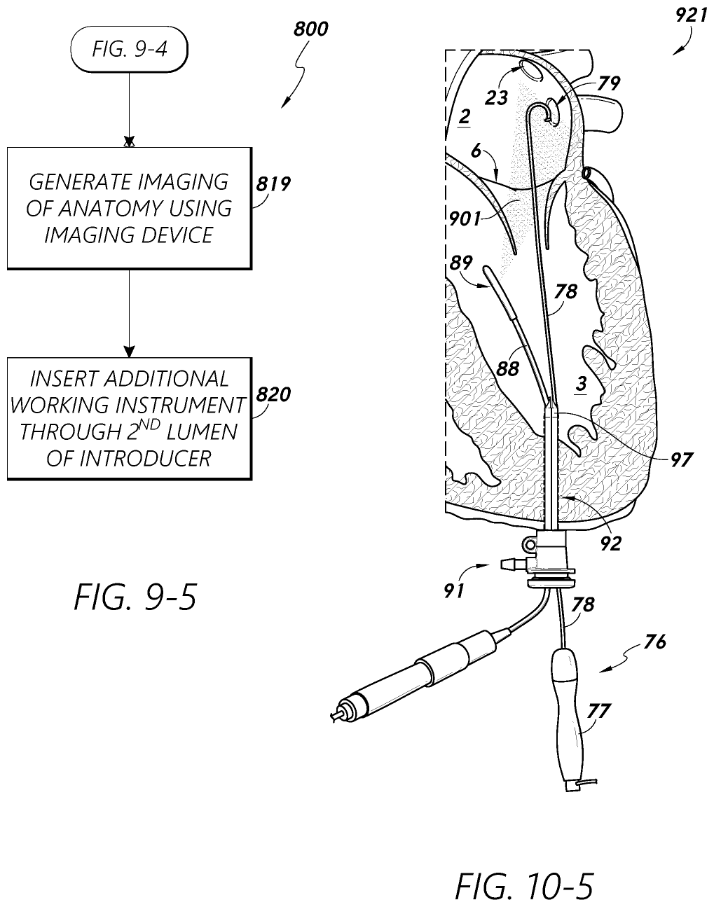

As indicated in block 812 of FIG. 9-3, the multi-lumen introducer 91 may be inserted with a dilator 95 projecting from a channel thereof. For example, the introducer 91 may include a central dilator port/channel through which a dilator may be inserted. The dilator 95 may further facilitate the insertion of the sheath 92 of the introducer 91 in such a manner as to reduce the risk of injury or damage to the cardiac tissue. In some examples, the introducer 91 and/or dilator 95 may be inserted over the guidewire 85 to thereby guide the sheath 92 into the ventricle 3. In some examples, a clamp-type structure/instrument 99 may be secured to the hub 94 of the introducer 91 to assist with placement of the introducer 91. In some implementations, the same dilator that is withdrawn in connection with block 811 may be inserted into the introducer 91 to aid placement of the introducer in connection with block 812, or the dilatory may remain in place (e.g., not removed in connection with block 811) prior to insertion of the introducer 91 over the dilator.

At block 814, the process 800 involves removing the dilator 95 from the introducer 91. At block 816, the process 800 involves securing the multi-lumen introducer 91 to the heart wall and/or other anatomical structure(s) and/or tissue. For example, the hub 94 of the introducer 91 may be secured using suturing and/or other attachment means or mechanism to tissue of the heart and/or chest of the patient. Securing the introducer 91 in place may serve to reduce the risk of damage from dislodgment thereof to the patient's anatomy. The guidewire 85 and dilator 95 can be removed while the introducer 91 maintains hemostasis, with or without a suitable delivery device inserted therein, throughout the procedure. In some implementations, once the introducer is properly placed, purse-string suture(s) can be tightened to reduce bleeding around the sheath 92 of the introducer.

At block 818, the process 800 involves inserting an elongate catheter 88 of an imaging device 86 in a first lumen/channel 96 of the introducer 91. For example, the imaging device 86 may comprise an intracardiac echocardiography catheter or other type of scope or imaging device. The imaging device 86 may include a distal imaging element 89, which may comprise an ultrasound transducer and/or other imaging element. In some examples, the catheter 88 may be inserted using a handle 87, which may be advanced distally with respect to the axis of the sheath 92 of the introducer 91.

The tip 89 of the catheter 88 may be maneuvered, tilted, retro-flexed, rotated, and/or actuated or manipulated in another manner to allow for imaging of the left ventricle 3 and/or left atrium 2. In some implementations, the imaging frequency may be adjusted to optimize penetration depth with a relatively clear resolution for viewing the left atrium 2 and pulmonary vein(s) 23 and/or the valvular and sub-valvular structures of the left ventricle 3.

At block 819, the process 800 involves generating imaging of internal cardiac anatomy using the imaging device 86. For example, the imaging may represent two-dimensional or three-dimensional images of one or more leaflets of a cardiac valve 6 and/or aspects associated with atrial tissue, such as left atrial tissue at or around one or more pulmonary veins 23.

At block 820, the process 800 involves inserting an additional working instrument 76 through a second lumen 97 of the sheath 92 of the multi-lumen introducer 91, wherein the instrument 76 includes a catheter or other elongate component 78 that may be advanced into the ventricle 3 and/or atrium 2 for execution of a medical procedure at least partially within an imaging window 901 of the imaging device 86. For example, as described in connection with various examples herein, the additional working instrument may comprise an ablation catheter or a shaft of a leaflet-tethering valve repair device, or any other type of working instrument.

In some implementations, once the imaging catheter 88 is in place, the ablation catheter 78 can be inserted through the channel 97 of the introducer sheath 92. The ablation catheter 78 may be guided to advance through the ventricle 3 and into the left atrium 2 through the mitral valve 6. In some implementations, the atrial structures (e.g., pulmonary veins 23) are identified through imaging, wherein the ablation catheter tip 79 is subsequently positioned and manipulated to target tissue for ablation near/around the pulmonary vein(s) 23.

In some implementations, the process 800 may further involve withdrawing the working instrument 76 (e.g., ablation catheter) from the lumen 97 of the introducer 91 and inserting in its place a leaflet repair shaft as described in detail herein for implementation of a valve repair procedure involving the deployment of one or more tissue anchors in one or more leaflets of the valve 6 and tethering such anchors to one or more other structures of the heart and/or patient anatomy. For example, following ablation, a valve repair device shaft may be inserted and advanced inside the ventricle 3 under continued imaging guidance from the imaging device 86 and/or in combination with transesophageal echocardiography to guide the deployment of one or more tissue anchors in the leaflet(s) of the valve 6. In some implementations, heart valve repair may occur prior to ablation. That is, the lumen 97 may be first utilized for valve repair, wherein after completion of the valve repair procedure, the shaft of the valve repair device is removed from the lumen and an ablation catheter is inserted in its place while the imaging device remains disposed within the first lumen 96 of the multi-lumen introducer 91.

Although certain procedures are described herein are with reference to repairing a cardiac mitral valve or tricuspid valve by the implantation of one or more leaflet anchors and associated cord(s), the methods presented are readily adaptable for various types of tissue, leaflet, and annular repair procedures. The methods described herein, for example, can be performed to selectively approximate two or more portions of tissue to limit a gap between the portions. That is, in general, the methods herein are described with reference to a mitral valve but should not be understood to be limited to procedures involving the mitral valve.

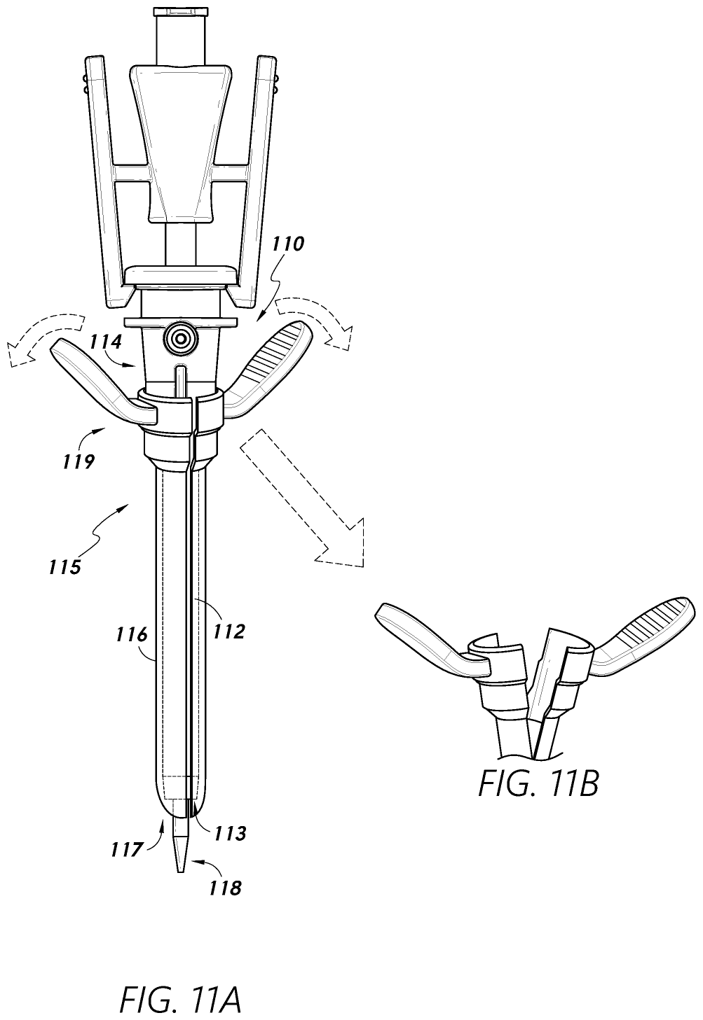

FIGS. 11A and 11B show a multi-lumen introducer device 114 implemented with a tearaway sheath 119 in accordance with one or more examples of the present disclosure. The multi-lumen introducer device no may have a hub portion 114 and a sheath portion 112, as described in detail herein. The introducer no may be utilized during one or more stages of an introduction process in which the introducer no is placed at least partially within an in internal chamber of the heart through the chest wall and ventricle wall together with a breakaway sheath covering 115, which may include a sheath portion 116 configured to be placed in a manner so as to cover the distal end 113 of the sheath 112 of the introducer 110. The distal end 117 of the tear-away sheath 115 can reduce the risk of tissue damage during insertion of the multi-lumen introducer 110. In some examples, the combination of the introducer 110 with the tear-away sheath 115 may be inserted with a dilator tip 118 projecting from a distal end thereof, wherein the distal end 117 of the tearaway sheath 115 may be configured and/or dimensioned to wrap around the dilator in a manner so as to reduce tissue damage. Once the distal end 113 of the introducer 110 is disposed within the target implantation site (e.g., left ventricle), the hub portion 119 may be broken apart to allow for removal of the tear-away sheath 115 from around the sheath 112 of the introducer 110, as shown in FIG. 11B.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure and appended claims should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An introducer device comprising:
a proximal hub including a first port and a second port;
first and second fluid valves associated with the first port and the second port, respectively;
a first elongate lumen projecting distally from the proximal hub, the first elongate lumen being in fluid communication with the first port; and
a second elongate lumen projecting distally from the proximal hub, the second elongate lumen being in fluid communication with the second port and fluidly isolated from the first elongate lumen.

2. The introducer device of claim 1, wherein the first elongate lumen and the second elongate lumen are part of an elongate access sheath structure.

3. The introducer device of claim 1, further comprising an outer tube surrounding at least a portion of the first elongate lumen and the second elongate lumen.

4. The introducer device of claim 1, wherein the proximal hub further includes a third port.

5. The introducer device of claim 4, wherein:
the first and second ports are configured to receive catheters; and
the third port is configured to receive a dilator.

6. The introducer device of claim 1, wherein:
the proximal hub further includes a proximal flange and a valve seal housing;
the valve seal housing houses the first and second fluid valves; and
the first and second elongate lumens project distally from the valve seal housing.

7. The introducer device of claim 1, wherein the proximal hub further includes a fluid inlet port.

8. The introducer device of claim 1, wherein the first and second elongate lumens are fluidly isolated from one another.

9. The introducer device of claim 1, wherein distal openings of the first and second elongate lumens are tapered towards one another.

10. The introducer device of claim 1, further comprising:
a first elongate shaft that forms the first elongate lumen;
a second elongate shaft that forms the second elongate lumen; and
an outer tube disposed around a length of the first and second elongate lumens.

11. An introducer device comprising:
a proximal hub including a first valved port and a second valved port; and
an elongate access sheath structure that projects distally from the proximal hub, the elongate access sheath structure comprising:
a first elongate lumen in fluid communication with the first port; and
a second elongate lumen in fluid communication with the second port and fluidly isolated from the first elongate lumen.

12. The introducer device of claim 11, wherein the elongate access sheath structure comprises:
a first elongate shaft forming the first elongate lumen; and
a second elongate shaft forming the second elongate lumen.

13. The introducer device of claim 12, wherein the elongate access sheath structure further comprises an outer tube, at least a portion of the first elongate shaft and the second elongate shaft being disposed within the outer tube.

14. The introducer device of claim 13, wherein the first and second elongate shafts extend past a distal end of the outer tube.

15. The introducer device of claim 14, wherein distal ends of the first and second elongate shafts are tapered towards a central axis of the outer tube.

16. The introducer device of any of claim 11, wherein the proximal hub further comprises a third valved port.

17. The introducer device of claim 16, wherein the third valved port is positioned about a central axis of the elongate access sheath structure.

18. The introducer device of claim 11, wherein the elongate access sheath structure has an oval cross-sectional shape.

19. The introducer device of claim 11, further comprising a tear-away sheath covering a distal end of the elongate access sheath structure.

20. The introducer device of claim 11, wherein the first and second valved ports include respective hemostatic fluid valves.
